(19) United States
(12) Patent Application Publication
(10) Pub. No.: US 2019/0060300 A1
(43) Pub. Date: Feb. 28, 2019

(54) Self-Emulsifying Compositions of CB2 Receptor Modulators

(71) Applicant: Sharon Anavi-Goffer, Oranit (IL)

(72) Inventor: Sharon Anavi-Goffer, Oranit (IL)

(21) Appl. No.: 16/081,105

(22) PCT Filed: Mar. 3, 2017
(86) PCT No.: PCT/IB17/00266

§ 371 Date: Aug. 30, 2018

Related U.S. Application Data
(60) Provisional application No. 62/303,494, filed on Mar. 4, 2016.
(60) Provisional application No. 62/303,508, filed on Mar. 4, 2016.

(51) Int. Cl. A61K 31/4515; A61P 25/14; A61K 31/015; A61K 31/05; A61P 25/18; A61K 31/09; A61K 31/355; A61K 31/375; A61K 31/519; A61K 47/34; A61K 9/00; A61K 9/28; A61K 9/50; A61K 31/337

(52) U.S. Cl. CPC A61K 31/4515; A61K 2300/00; A61K 31/015; A61K 31/05; A61P 25/18; A61K 31/09; A61K 31/355; A61K 31/375; A61K 31/519; A61K 47/34; A61K 9/0024; A61K 9/2846; A61K 9/5031; A61K 31/337; A61P 25/14

(57) ABSTRACT

Disclosed are stable self-emulsifying compositions comprising at least one CB2 receptor modulator, a self-emulsifying vehicle and optionally at least one antipsychotic agent for use in the treatment of mental disorders, methods of preparing such compositions and methods of treating mental disorders using same. Disclosed are also stable self-emulsifying compositions comprising beta caryophyllene (BCP) or HO-308 as sole active agent or in combination with humulene, an antipsychotic for use in the treatment of schizophrenia, methods of making such compositions and methods of treating schizophrenia rising BCP. Disclosed are also stable self-emulsifying compositions comprising 4-0-methylhonokiol (MH) as sole active agent or in combination with caryophyllene oxide, and optionally at least one antipsychotic agent for use in the treatment of tic disorders, methods of making such compositions and methods of treating Tourette syndrome using MH

### CROSS REFERENCE TO RELATED APPLICATIONS

This application claims priority to U.S. Provisional Patent Application Ser. No. 62/303,508, filed on Mar. 4, 2016, and to Provisional Patent Application Ser. 62/303,494 filed on Mar. 4, 2016, the entire contents of each of which are hereby incorporated by reference in their entirety.

### FIELD OF THE INVENTION

The present invention is in the field of pharmaceutical compositions and discloses novel compositions for the oral administration of Cannabinoid Receptor Type 2 (CB2) modulators and optionally of an antipsychotic agent for the treatment of mental disorders.

### BACKGROUND

Mental disorders can arise from multiple sources and affect a large percentage of the population. There are a range of different types of treatment of mental disorders and what is most suitable depends on the disorder and on the individual.

Schizophrenia is a mental disorder which affects about 1% of the population (Lewis & Lieberman, 2000), and genetic and environmental factors underlie the eventual eruption of the disease (Ross, 2006). Schizophrenia is often chronic, characterized by deterioration of social contact, cognitive deficits, anxiety and depression, resulting in suicide in about 10% of the schizophrenic population (Lewis & Lieberman, 2000).

Another important mental disorder is tic disorders, specifically, Tourette syndrome (TS), which is characterized by multiple motor tics and at least one vocal tic. Starting at childhood, TS includes ties like blinking, coughing, throat clearing, sniffing and facial movements. About 1% of the school-age children and adolescents have Tourette's.

### SUMMARY

Aspects of the invention relate to stable self-emulsifying compositions comprising at least one CB2 modulator, a self-emulsifying vehicle and optionally at least one additional antipsychotic agent, methods of making the compositions and methods of treatment using same for the treatment of mental disorders.

According to aspects illustrated therein, there is provided stable self-emulsifying compositions comprising a therapeutically effective amount of at least one CB2 receptor modulator in substantially pure form, a self-emulsifying vehicle and optionally a therapeutically effective amount of at least one antipsychotic agent, for use in treating a mental disorder in a patient in need thereof. In some embodiments, the self-emulsifying (or self-emulsifiable) drug delivery systems (SEDDS) can be liquid compositions generally used for oral delivery, or more particularly designed for improved delivery of drug moieties with poor aqueous solubility (see Nagaraju J. Seminar, M. Pharm. II Sem. 2010, Kakatiya University, Warangal, Department of Pharmaceutics, University College of Pharmaceutical Sciences.

According to aspects of the invention, the self-emulsifying drug delivery system (SEDDS) compositions enable to reduce the oral dose to correspond to the dose given by intraperitoneal injections or a lower dose.

According to other aspects of the invention, the self-emulsifying drug delivery system (SEDDS) compositions potentiate the therapeutic actions of a CB2 receptor modulator, reducing the required dose hence its toxicity.

According to aspects of the invention, the compositions of this invention can be formulated as a stable self-emulsifying drug delivery system (SEDDS) comprising at least one CB2 receptor modulator, optionally at least one antipsychotic agent and a self-emulsifying vehicle comprising at least one oil, at least one surfactant with HLB<9, at least one surfactant with HLB>13, at least one co-surfactant and at least one antioxidant and/or free-radical scavenger. The antioxidant and/or free-radical scavenger can be selected from vitamin E, d-alpha-tocopherol (1-10% w/w), dl-alpha-tocopherol (2-15% w/w), dl-alpha-tocopheryl acetate (2-15% w/w), mixed tocopherols (alpha, beta, gama—1-10% w/w), d-alpha-tocopherol acetate (2-15% w/w), butylated hydroxyanisole (BHA, 0.01-0.5% w/w), tocophersolan (TPGS, tocopherol PEG ester succinate) (2-10% w/w), vitamin C, beta-carotene, butylated hydroxy toluene, butylated hydroxyanisole or other FDA-approved antioxidant listed in the FDA's Inactive Ingredients Database (IID), and combinations thereof.

In some embodiments, the ratio of antioxidant/CB2 modulator, such as but not limited to BCP, is from 1:1 to 2:1 w/w. In some embodiments, the antioxidant/CB2 modulator is from 1:1 to 3:1 w/w. In some embodiments, the ratio of antioxidant/CB2 modulator is from 1:1 to 4:1 w/w. In some embodiments, the ratio of antioxidant/CB2 modulator is from 1:1 to 5:1 w/w. In some embodiments, the ratio of antioxidant/CB2 modulator is from 2:1 to 3:1 w/w. In some embodiments, the ratio of antioxidant/CB2 modulator is from 2:1 to 4:1 w/w. In some embodiments, the ratio of antioxidant/CB2 modulator is from 2:1 to 5:1 w/w. In some embodiments, the ratio of antioxidant/CB2 modulator is from 3:1 to 4:1 w/w. In some embodiments, the ratio of antioxidant/CB2 modulator is from 3:1 to 5:1 w/w. In some embodiments, the ratio of antioxidant/CB2 modulator is from 1:1 to 10:1w/w. In some embodiments, the ratio of antioxidant/CB2 modulator is from 2:1 to 10:1 w/w. In some embodiments, the ratio of antioxidant/CB2 modulator is from 3:1 to 10:1 w/w. In some embodiments, the ratio of antioxidant/CB2 modulator is from 4:1 to 10:1 w/w. In some embodiments, the ratio of antioxidant/CB2 modulator is from 5:1 to 10.1 w/w. In some embodiments, the ratio of antioxidant/CB2 modulator is from 6:1 to 10:1 w/w. In some embodiments, the ratio of antioxidant/CB2 modulator is from 7:1 to 10:1 w/w. In some embodiments, the ratio of antioxidant/CB2 modulator is from 8:1 to 10:1w/w. In some embodiments, the ratio of antioxidant/CB2 modulator is from 9:1 to 10:1 w/w. In some embodiments, the ratio of antioxidant/CB2 modulator is from 5:1 to 15:1 w/w. In some embodiments, the ratio of antioxidant/CB2 modulator is from 5:1 to 20:1 w/w. In some embodiments, the ratio of antioxidant/CB2 modulator is from 5:1 to 25:1 w/w. In some embodiments, the ratio of antioxidant/CB2 modulator is from 5:1 to 30:1 w/w. In some embodiments, the ratio of antioxidant/CB2 modulator is from 5:1 to 35:1 w/w. In some embodiments, the ratio of antioxidant/CB2 modulator is from 5:1 to 40:1 w/w. In some embodiments, the ratio of antioxidant/CB2 modulator is from 10:1 to 15:1 w/w. In some embodiments, the ratio of antioxidant/CB2 modulator is from 10:1 to 20:1 w/w. In some embodiments, the ratio of antioxidant/CB2 modulator is from 10:1 to 25:1 w/w. In some embodiments, the ratio of antioxidant/CB2 modulator is from 10:1 to 30:1 w/w. In some embodiments, the ratio of antioxidant/CB2 modulator is from 10:1 to 35:1 w/w. In some embodiments, the ratio of antioxidant/CB2 modulator is from 10:1 to 40:1 w/w. In some embodiments, the ratio of antioxidant/CB2 modulator is from 15:1 to 20:1 w/w. In some embodiments, the ratio of antioxidant/CB2 modulator is from 15:1 to 25:1 w/w. In some embodiments, the ratio of antioxidant/CB2 modulator is from 15:1 to 30:1 w/w. In some embodiments, the ratio of antioxidant/CB2 modulator is from 15:1 to 35:1 w/w. In some embodiments, the ratio of antioxidant/CB2 modulator is from 1.51 to 40:1 w/w. In some embodiments, the ratio of antioxidant/CB2 modulator is from 20:1 to 25:1 w/w. In some embodiments, the ratio of antioxidant/CB2 modulator is from 20:1 to 30:1 w/w. In some embodiments, the ratio of antioxidant/CB2 modulator is from 20:1 to 35:1 w/w. In some embodiments, the ratio of antioxidant/CB2 modulator is from 20:1 to 40:1 w/w. In some embodiments, the ratio of antioxidant/CB2 modulator is from 25:1 to 30:1 w/w. In some embodiments, the ratio of antioxidant/CB2 modulator is from 25:1 to 35:1 w/w. In some embodiments, the ratio of antioxidant/CB2 modulator is from 25:1 to 40:1 w/w. In some embodiments, the ratio of antioxidant/CB2 modulator is from 30:1 to 35:1 w/w. In some embodiments, the ratio of antioxidant/CB2 modulator is from 30:1 to 40:1 w/w. In some embodiments, the above composition can spontaneously form an oil-in-water emulsion upon dilution with water containing media or body fluid.

In some embodiments, the ratio of antioxidant/CB2 modulator, such as but not limited to 4-0-methylhonokiol (MH), is from 40:1 to 2500:1 w/w. In some embodiments, the antioxidant/CB2 modulator is from 40:1 to 80:1 w/w. In some embodiments, the ratio of antioxidant/CB2 modulator is from 40:1 to 100:1 w/w. In some embodiments, the ratio of antioxidant/CB2 modulator is from 100:1 to 500:1 w/w. In some embodiments, the ratio of antioxidant/CB2 modulator is from 500:1 to 1000:1 w/w. In some embodiments, the ratio of antioxidant/CB2 modulator is from 1000:1 to 1500:1 w/w. In some embodiments, the ratio of antioxidant/CB2 modulator is from 1500:1 to 2000:1 w/w. In some embodiments, the ratio of antioxidant/CB2 modulator is from 2000:1 to 2500:1 w/w. In some embodiments, the ratio of antioxidant/CB2 modulator is from 3:1 to 5:1 w/w. In some embodiments, the ratio of antioxidant/CB2 modulator is from 40:1 to 100:1 w/w. In some embodiments, the ratio of antioxidant/CB2 modulator is from 40:1 to 50:1 w/w. In some embodiments, the ratio of antioxidant/CB2 modulator is from 40:1 to 60:1 w/w. In some embodiments, the ratio of antioxidant/CB2 modulator is from 40:1 to 80:1 w/w. In some embodiments, the ratio of antioxidant/CB2 modulator is from 60:1 to 500:1 w/w. In some embodiments, the ratio of antioxidant/CB2 modulator is from 80:1 to 500:1 w/w. In some embodiments, the ratio of antioxidant/CB2 modulator is from 100:1 to 500:1 w/w. In some embodiments, the ratio of antioxidant/CB2 modulator is from 150:1 to 250:1 w/w. In some embodiments, the ratio of antioxidant/CB2 modulator is from 150:1 to 280:1 w/w. In some embodiments, the ratio of antioxidant/CB2 modulator is from 150:1 to 300:1 w/w. In some embodiments, the ratio of antioxidant/CB2 modulator is from 200:1 to 500:1 w/w. In some embodiments, the ratio of antioxidant/CB2 modulator is from 300:1 to 500:1 w/w. In some embodiments, the ratio of antioxidant/CB2 modulator is from 400:1 to 500:1 w/w. In some embodiments, the ratio of antioxidant/CB2 modulator is from 600:1 to 1000:1 w/w. In some embodiments, the ratio of antioxidant/CB2 modulator is from 700:1 to 1000:1 w/w. In some embodiments, the ratio of antioxidant/CB2 modulator is from 800:1 to 1000:1 w/w. In some embodiments, the ratio of antioxidant/CB2 modulator is from 900:1 to 1000:1 w/w. In some embodiments, the ratio of antioxidant/CB2 modulator is from 1000:1 to 1200:1 w/w. In some embodiments, the ratio of antioxidant/CB2 modulator is from 1000:1 to 1300:1 w/w. In some embodiments, the ratio of antioxidant/CB2 modulator is from 1000:1 to 1400:1 w/w. In some embodiments, the ratio of antioxidant/CB2 modulator is from 1200:1 to 1400:1 w/w. In some embodiments, the ratio of antioxidant/CB2 modulator is from 1200:1 to 1500:1 w/w. In some embodiments, the ratio of antioxidant/CB2 modulator is from 1300:1 to 1500:1 w/w. In some embodiments, the ratio of antioxidant/CB2 modulator is from 1400:1 to 1500:1 w/w. In some embodiments, the ratio of antioxidant/CB2 modulator is from 1500:1 to 1600:1 w/w. In some embodiments, the ratio of antioxidant/CB2 modulator is from 1500:1 to 1700:1 w/w. In some embodiments, the ratio of antioxidant/CB2 modulator is from 1500:1 to 1800:1 w/w. In some embodiments, the ratio of antioxidant/CB2 modulator is from 1500:1 to 1700:1 w/w. In some embodiments, the ratio of antioxidant/CB2 modulator is from 1500:1 to 1800:1 w/w. In some embodiments, the ratio of antioxidant/CB2 modulator is from 1500:1 to 1900:1 w/w. In some embodiments, the ratio of antioxidant/CB2 modulator is from 1500:1 to 2000:1 w/w. In some embodiments, the ratio of antioxidant/CB2 modulator is from 1600:1 to 2000:1 w/w. In some embodiments, the ratio of antioxidant/CB2 modulator is from 1700:1 to 2000:1 w/w. In some embodiments, the ratio of antioxidant/CB2 modulator is from 1800:1 to 2000:1 w/w. In some embodiments, the ratio of antioxidant/CB2 modulator is from 2000:1 to 2200:1 w/w. In some embodiments, the ratio of antioxidant/CB2 modulator is from 2000:1 to 2300:1 w/w. In some embodiments, the ratio of antioxidant/CB2 modulator is from 2000:1 to 2400:1 w/w. In some embodiments, the ratio of antioxidant/CB2 modulator is from 2000:1 to 2500:1 w/w. In some embodiments, the ratio of antioxidant/CB2 modulator is from 2100:1 to 2500:1 w/w. In some embodiments, the ratio of antioxidant/CB2 modulator is from 2200:1 to 2500:1 w/w. In some embodiments, the ratio of antioxidant/CB2 is from 2300:1 to 2500:1 w/w. In some embodiments, the ratio of antioxidant/CB2 is from 2400:1 to 2500:1 w/w. In some embodiments, the above composition can spontaneously form an oil-in-water emulsion upon dilution with water containing media or body fluid.

Some aspects of the invention relate to compositions comprising CB2 receptor selective or highly selective agonists as sole active, methods of making the compositions and methods using CB2 receptor selective agonists for the treatment of mental disorders. In some embodiments, the CB2:CB1 Ki ratio for high affinity ligands with Ki 1-50 nM ratio is about 1:500 while the CB2:CB1 Ki for low affinity ligands with Ki 50-200 nM ratio is about 1:50.

Some aspects of the invention relate to compositions comprising as CB2 receptor selective agonist beta-caryophyllene (BCP) and optionally at least one antipsychotic, agent in the vehicle of a self-emulsifying drug delivery system (SEDDS vehicle), methods of making the compositions and methods using the compositions for the treatment of mental disorders. Some aspects of the invention, relate to compositions comprising as CB2 receptor selective agonist beta-caryophyllene (BCP) and optionally at least one antipsychotic agent in a SEDDS vehicle, methods of making the compositions and methods using fee compositions for the treatment of mental disorders.

Some other aspects of the invention relate to compositions comprising as CB2 receptor selective agonist [(1R,2R,5R)-2-[2,6-dimethoxy-4-(2-methyloctan-2-yl) phenyl]-7,7-dimethyl-4-bicyclo[3.1.1]hept-3-enyl] methanol (HU-308) and optionally at least one antipsychotic agent in the vehicle of a self-emulsifying drug delivery system (SEDDS), methods of making the compositions and methods using the compositions for the treatment of mental disorders.

Some other aspects of the invention relate to compositions comprising as CB2 receptor selective agonist [(1R,2R,5R)-2-[2,6-dimethoxy-4-(2-methyloctan-2-yl)phenyl]-7,7-dimethyl-4-bicyclo [3.1.1]hept-3-enyl] methanol (HU-308) and optionally at least one antipsychotic agent in a SEDDS vehicle, methods of making the compositions and methods using the compositions for the treatment of mental disorders. In some embodiments, the mental disorder is schizophrenia. In some embodiments, the schizophrenia is selected from the group consisting of paranoid schizophrenia, disorganized schizophrenia, undifferentiated schizophrenia, catatonic schizophrenia and residual schizophrenia. It should be appreciated that onset of schizophrenia can occur at any age, infancy, childhood, adolescence or adulthood.

According to some aspects of the invention, the method of treatment comprises treating at least one symptom of schizophrenia selected from the group consisting of a negative symptom of schizophrenia, and/or a positive symptom of schizophrenia, both positive and negative symptoms as well as other symptoms of schizophrenia (e.g. cognitive symptoms).

In some aspects, the composition is formulated as an orally-administrable dosage form. The oral composition is formulated in a dosage form selected from the group consisting of a capsule, a liquid composition for oral administration, a syrup, a suspension, an emulsion and an ingestible solution.

In other aspects, the composition can be a topical composition. In some embodiments, the topical composition can be formulated as a transdermal gel, cream, patch or topical spray.

In some aspects of the invention, the composition comprises at least one CB2 receptor modulator, a self-emulsifying vehicle and optionally a therapeutically effective amount of at least one additional active agent selected from the group consisting of an antipsychotic agent, a GPR55 modulator, at least one cognitive enhancer, at least one anti-diabetic agent, an anti-inflammatory agent, an enzyme enhancer, an enzyme inhibitor, an antidepressant, an anxiolytic, a terpene/terpenoid combinations thereof.

In some aspects of the invention, the composition can further comprise at least one enzyme modulator selected from the group targeting the enzymes cyclooxygenase-2 (COX-2), fatty acid amide hydrolase (FAAH), monoacylglycerol lipase (MGL), α/β-hydrolase domain containing 6 (ABDH6 or ABDH6), α/β-hydrolase domain containing 12 (ABDH12), α/β-hydrolase domain containing 4 (ABDH4), sn-1-diacylglycerol lipase alpha (DAGLalpha), sn-1-diacylglycerol lipase beta (DAGLbeta), N-acyl phosphatidylethanolamine phospholipase D (NAPE-PLD), phosphodiesterase 1 (GDE1), phospholipase C (PLC), phospholipase D (PLD) and combination thereof.

In some aspects of the invention, the composition can further comprise at least one antipsychotic agent. The at least one antipsychotic agent can be selected from the group consisting of benperidol, bromperidol, droperidol, haloperidol, timiperone, fluspirilene, penfluridol, pimozide, acepromazine, chlorpromazine, cyamemazine, dixyrazine, fluphenazine, levomepromazine, mesoridazine, perazine, pericyazine, perphenazine, pipotiazine, prochlorperazine, promazine, promethazine, prothipendyl, thioproperazine, thioridazine, trifluoperazine, triflupromazine, chlorprothixene, clopenthixol, flupentixol, thiothixene, zuclopenthixol, amisulpride, amoxapine, aripiprazole, dehydroaripiprazole, asenapine, cariprazine, clozapine, blonanserin, iloperidone, lurasidone, melperone, nemonapride, olanzapine, paliperidone, paliperidone palmitate, perospirone, quetiapine, remoxipride, risperidone, sertindole, sultopride, trimipramine, ziprasidone, brexpiprazole, ITI-007, pimavanserin, RP5063 (RP5000) cannabidiol (CBD), cannabidivarin (CBDV), cannabiodiolic acid (CBDA), tetrahydrocannabivarin (THCV), OPC-14857, DM-1458, DM-1451, DM-4452, DM-1454, DCPP, cannabigerol (CBG) and its analogs CBGA and CBGV and combinations thereof.

According to some aspects of the invention, there is also provided the use of beta-caryophyllene (BCP) as sole active agent in a self-emulsifying vehicle in the manufacture of a composition (also known as a medicament) for treating schizophrenia in a subject in need thereof. In some aspects, the composition is formulated for use in the treatment of a human subject. In some other aspects, the composition is formulated for use in the treatment of a non-human subject

In some aspects, the schizophrenia is selected from the group consisting of paranoid schizophrenia, disorganized schizophrenia, undifferentiated schizophrenia, catatonic schizophrenia and residual schizophrenia.

In some aspects of the invention, the at least one antipsychotic agent can be co-administered in a single dosage form together with the CB2 receptor modulator. In some other aspects, the at least one antipsychotic agent can be co-administered in a dosage form separate from the CB2 receptor modulator. The co-administration can comprise sequential or simultaneous administration. In some embodiments, the sequential administration comprises administration of the at least one antipsychotic agent prior to administration of the CB2 receptor modulator or subsequent to administration of the CB2 receptor modulator.

According to some aspects of the invention, the at least one CB2 receptor modulator in the composition of the present disclosure is selected from the group consisting of at least one CB2 receptor agonist or partial-agonist, at least one CB2 receptor antagonist or inverse agonist, at least one CB2 receptor antagonist or inverse agonist which is also a selective estrogen receptor modulator (SERM), at least one type of CB2 receptor allosteric modulator and combinations thereof.

According to some aspects of the invention, BCP can be one of the CB2 receptor selective agonists of this invention.

In some aspects, the BCP used for implementing the teachings herein is at least about 65%, at least about 75%, at least about 85% and even at least about 95% by weight E-BCP. In some embodiments, the BCP is substantially pure (at least about 98% or about 99% by weight) E-BCP.

In other aspects, the BCP used for implementing the teachings herein is at least about 65%, at least about 75%, at least about 85% and even at least about 95% by weight Z-BCP. In some embodiments, the BCP is substantially pure (at least about 98% or about 99% by weight) Z-BCP.

In some aspects, the BCP used for implementing the teachings herein is at least about 65%, at least about 75%, at least about 85% and even at least about 95% or about 98% by weight E-BCP and/or Z-BCP. In some embodiments, the BCP is substantially pure (at least about 97-99% by weight) E-BCP and/or Z-BCP.

For example, in some aspects, the BCP used for implementing the teachings herein comprises at least about 49% E-BCP, about 1-49% Z-BCP, about 1-5% BCP oxide and about 1-15% alpha humulene.

For example, in some aspects, the BCP used for implementing the teachings herein comprises about 45-49% E-BCP, about 45-49% Z-BCP, about 1-5% BCP oxide and about 1-5% alpha humulene.

For example, in some aspects BCP used for implementing the teachings herein comprises about 45-90% E-BCP, about 5-30% Z-BCP, about 1-5% BCP oxide and traces alpha humulene.

According: to an aspect of the invention, there is also provided a composition comprising a CB2 receptor selective agonist and a self-emulsifying vehicle for use in treating schizophrenia.

According to an aspect of the invention, there is also provided a use of a composition comprising a CB2 receptor selective agonist and a self-emulsifying vehicle in the manufacture of a composition for treating schizophrenia in a subject in need thereof.

According to an aspect of the invention, there is also provided a method for the treating schizophrenia in a subject in need thereof, the method comprising administering a therapeutic composition comprising a CB2 receptor selective agonist in a self-emulsifying vehicle.

### BRIEF DESCRIPTION OF THE FIGURES

Some embodiments of the invention are described herein with reference to the accompanying figures. The description, together with the figures, makes apparent to a person having ordinary skill in the art how some embodiments of the invention may be practiced. The figures are for the purpose of illustrative discussion and no attempt is made to show structural details of an embodiment in more detail than is necessary for a fundamental understanding of the invention. For the sake of clarity, some objects depicted in the figures are not to scale.

FIG. 1 shows results demonstrating that oral treatment with BCP in self-emulsifying oral formulation at adolescence reversed the effect of PCP on mice in the forced-swim test.

FIG. 2 shows that oral treatment with treatment with BCP in self-emulsifying oral formulation at adolescence reversed the effect of PCP on activity of mice in the open field test.

FIGS. 3A-B show results demonstrating that oral treatment with BCP in self-emulsifying oral formulation at adolescence reversed the effect of PCP on mice in the social interaction test (FIG. 3A) but did not affect their body weight (FIG. 3B).

FIG. 4 shows results demonstrating that oral treatment with BCP in oil at adolescence did not reverse the effect of PCP on mice in the forced-swim test.

### DETAILED DESCRIPTION

Unless otherwise defined, all technical and scientific terms used herein have the same meaning as commonly understood by one of ordinary skill in the art to which the invention pertains. In case of conflict, the specification, including definitions, takes precedence.

As used herein, the terms “comprising”, “including”, “having” and grammatical variants thereof are to be taken as specifying the stated features, integers, steps or components but do not preclude the addition of one or more additional features, integers, steps, components or groups thereof.

As used herein, the indefinite articles “a” and “an” mean “at least one” or “one or more”unless the context clearly dictates otherwise.

As used herein, when a numerical value is preceded by the term “about”, the term “about” is intended to indicate +/−10%.

As used herein, the term “treating” or “treatment” includes curing a condition, treating a condition, preventing a condition, treating symptoms of a condition, curing symptoms of a condition, ameliorating symptoms of a condition, treating effects of a condition, ameliorating effects of a condition, and preventing results of a condition.

As used herein a “therapeutic composition” refers to a preparation of one or more of the active ingredients with other components such as pharmaceutically-acceptable carriers and excipients. The purpose of a therapeutic composition is to facilitate administration of an active ingredient to a subject.

The term “pharmaceutically acceptable carrier” or “self-emulsifying vehicle” refers to a carrier or a diluent that does not cause significant irritation to a subject, effectively provides the active agent(s) to the patient in need thereof and does not substantially abrogate the activity and properties of the administered active ingredients. An adjuvant is included under these phrases. The term “excipient” refers to an inert substance added to a therapeutic composition to further facilitate administration of an active ingredient.

Therapeutic compositions used in implementing the teachings herein may be formulated using techniques with which one of average skill in the art is familiar in a conventional manner using one or more pharmaceutically-acceptable carriers comprising excipients and adjuvants, which facilitate processing of the active ingredients into a pharmaceutical composition and generally includes mixing an amount of the active ingredients with the other components. Suitable techniques are described in “Remington's Pharmaceutical Sciences,” Mack Publishing Co., Easton, Pa., latest edition, which is incorporated herein by reference. For example, pharmaceutical compositions useful in implementing the teachings herein may be manufactured by one or more processes that are well known in the art, e.g., mixing, blending, homogenizing, dissolving, granulating, emulsifying, encapsulating, entrapping and lyophilizing processes.

The “Hydrophilic Lipophilic Balance” (HLB) system, the balance between the hydrophilic and lipophilic moieties of a surface-active molecule, is used as a basis for rational means of selecting and classifying emulsifying agents or surfactants. In the HLB system the surfactant is assigned a number between 1 and 20. Surfactants with HLB values of between 3 and 6 are lipophilic and form water-in-oil emulsions, while values, of 8 to 18 indicate predominantly hydrophilic characteristics and the formation of oil-in-water emulsions.

Pharmaceutical compositions suitable for implementing the teachings herein include compositions comprising active ingredients in an amount effective to achieve the intended purpose (a therapeutically effective amount). Determination of a therapeutically effective amount is well within the capability of those skilled in the art, for example, is initially estimated from animal models such as rats, mice, monkey or pigs.

The terms self-emulsifying and self-emulsifiable, as used herein, can be used interchangeably.

SEDDS is a broad term associated with the production of emulsions with a droplet size ranging from a few nanometers to several microns, which can be classified as self-micro-emulsifying drug delivery systems (SMEDDS) and self-nanoemulsifying drug delivery systems (SNEDDS) (Zanchetta B. et al. Adv. Chem. Eng. 2015, 5:3).

SEDDS formulation is a liquid composition. SEDDS are waterless compositions which upon dilution with water containing media or body fluid self-emulsify forming an oil-in-wafer emulsion. According to the specific composition and mode of preparation, SEDDS may form, upon dilution with aqueous media, emulsions with different droplet sizes.

The present invention provides a highly effective stable oral composition, comprising a therapeutically effective amount of at least one CB2 receptor selective or highly selective agonist in substantially pure form in a self-emulsifying vehicle and optionally a therapeutically effective amount of at least one antipsychotic agent in a self-emulsifying vehicle, for use in treating a mental disorder in a patient in need thereof. In the context of this disclosure, the term “selective” when used alone is meant genetically, meaning that it includes also highly selective CB2 receptor modulator. In some embodiments, the CB2:CB1 Ki ratio for high affinity ligands with Ki 1-50 nM ratio is about 1:500 while the CB2:CR1 Ki for low affinity ligands with Ki 50-200 nM ratio is about 1:50.

In some aspects of the invention, some of the CB2 receptor selective or highly selective agonists can be synthetic cannabinoids or cannabinoids of plant origin (phytocannabinoids) such as cannabis, hemp, cloves, malabatbrum, West African pepper, hops, oregano, etc.

The cannabinoids are a group of chemical compounds of very diverse structures.

The most important types of phytocannabinoids are: cannabigerol-type (CBG), cannabichromene-type (CBC), cannabidiol-type (CBD), tetrahydrocannabinol- and cannabinol-type (THC, CBN), cannabielsoin-type (CBE), iso-tetrahydrocannabinol-type (iso-THC), cannabicyclol-type (CBL), and cannabicitran-type (CBT). The most studied cannabinoids are THC, CBD, CBG and CBN. At least 85 different cannabinoids have been isolated from the cannabis plant. These compounds have very different affinities for the cannabinoid or non-cannabinoid receptors. Some are neutral ligands (no or very little affinity to the cannabinoid receptors), some are CB1 and CB2 receptor agonists, some are CB1 and CB2 receptor partial agonists, some are CB1 and CB2 receptor antagonists, some are CB1 and CB2 receptor inverse-agonists, some are combination thereof and only a few are specific and selective agonists or antagonists. Some cannabinoids (like CBD, CBDA, CBDV, CBG, CBGA, CBGV, THC and THCV) are inhibitors of the GPR55 ligand (Anavi-Goffer et al. 2012).

THC, THCV and CBN are non-selective CB1 and CB2 receptor ligands. In fact delta-9-THC is a weak CB1 and CB2 receptor partial agonist (Childers, 2006), thus that in the presence of a more potent selective agonist delta-9-THC will antagonize its effects. CBC, CBD, CBDV, CBDA, CBG, CBGV, CBGA, THCA and THCV have not been reported to activate the CB1 or CB2 receptors with significant potency (Handbook of Cannabis, Oxford University Press, R. G. Pertwee Editor, p. 137, 2014). Summing up, unlike the CB2 receptor selective agonists of this invention, none of the above cannabinoids are selective or highly selective CB2 receptor agonists.

Most of the commercially available cannabinoids are in fact loosely defined mixtures of a cannabinoid with other cannabinoids, impurities, geometrical isomers and enantiomers. The cannabinoid's proneness to spontaneous oxidation complicates even more the purity issue of these substances.

The affinities for two different cannabinoid receptors (CB1 and CB2 receptors) complicate the issue of pharmacological activity. Therefore, the present disclosure uses as active agents well-defined stable highly pure CB2 receptor selective agonists. Most of the CB2 receptor agonists of this invention are potent selective CB2 receptor agonists.

The mental disorder to be treated by the compositions and methods described herein can be selected from the group consisting of schizophrenia, schizoaffective disorder, bipolar disorder I and II, unipolar disorder, multiple personality disorder, psychotic disorders, depression. psychotic depression, depressive disorders, major depressive disorder, depression associated with tic disorders, epilepsy, anxiety disorders, autistic spectrum disorder, enuresis and addiction, Asperger syndrome, oppositional defiant disorder, behavioral disturbance, agitation, psychosis/agitation associated with Alzheimer's disease, psychosis associated with Parkinson's disease, personality disorders, borderline personality disorder, avoidant personality disorder, attention-deficit/hyperactive disorder (ADHD, ADD, HD), mania, dementia, anorexia, anorexia nervosa, anxiety, generalized anxiety disorder, social anxiety disorder, body dismographic disorder, obsessive compulsive disorder, paranoid disorder, nightmares, agitation, post-traumatic stress disorder (PTSD), severe mood dysregulation and Tourette's syndrome.

Some embodiments of the invention relate to compositions comprising at least one Cannabinoid Receptor Type 2 (CB2) receptor selective agonist as sole active, methods of making the compositions and methods using CB2 receptor selective agonists for the treatment of mental disorders. Some other embodiments relate to compositions comprising CB2 receptor selective agonists in combination with at least one antipsychotic agent in a self-emulsifying vehicle.

Other embodiments of the invention relate to compositions comprising beta-caryophyllene (BCP) as sole CB2 receptor selective agonist, methods of making the compositions and methods using BCP for the treatment of schizophrenia. The use of BCP in schizophrenia disclosed in this invention is unexpected and surprising, as cannabinoids are known to cause aggravation of psychosis in patients with schizophrenia. Thus, for example, TBC is known to induce a range of positive symptoms of schizophrenia (according to The Diagnostic and Statistical Manual of Mental Disorders (DSM)), and THC treated schizophrenic patients experienced an exacerbation of symptoms (Deepak Cyril D'Souza et al, Eur Arch Psychiatry Clin. Neurosci 2009 October; 259(7): 413-431). In addition, while THC can induce anxiety in some patients, BCP reduces anxiety.

When found in nature, BCP typically appears as a mixture of two isomers E-BCP and Z-BCP, together with substantially inactive sesquiterpenes such as alpha-humulene and derivatives Such as BCP oxide. Typically, natural sources include a greater proportion of E-BCP than Z-BCP.

For implementing the teachings herein, the BCP includes both E-BCP and Z-BCP, alone or in combination.

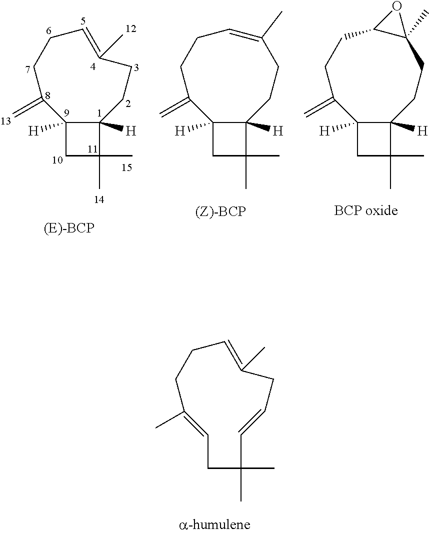

Some other embodiments of the invention relate to compositions comprising beta-caryophyllene (BCP) in combination with risperidone, paliperidone, paliperidone palmitate, aripiprazole, quetiapine, CBD and its analogs, THCV, brexpiprazole and combinations thereof, methods of making the compositions and methods using the compositions for the treatment of schizophrenia.

Other embodiments of the invention relate to compositions comprising beta-caryophyllene (BCP) as sole CB2 receptor selective agonist, methods of making the compositions and methods using BCP for the treatment of mental disorders other than schizophrenia. Other embodiments of the invention relate to compositions comprising beta-caryophyllene (BCP) as sole CB2 receptor selective agonist, methods of making the compositions and methods using BCP for the treatment of mental disorders other than schizophrenia, depression and anxiety.

The Cannabinoid Receptor Type 2 (CB2) is a guanine nucleotide-binding protein (G protein)-coupled receptor that in humans is encoded, by the CNR2 gene.

Recent studies have identified the cannabinoid CB2 receptor in the brain. Up-regulation of CB2 receptor expression in the brain during central nervous system pathologies has been demonstrated for certain neurological diseases.

In some embodiments, the CB2 receptor selective agonist in the compositions of this invention is selected from the group comprising BCP, [(1R,2R,5R)-2-[2, 6-dimethoxy-4-(2-methyloctan-2-yl)phenyl]-7,7-dimethyl-4-bicyclo[3.1.1]hept-3-enyl] methanol (HU-308), HU-433, HU-910, HU-914, CB 65, GP 1a, GP 2a, GW 405833, JWH 015, JWH 133, AM1241, L-759,656, L-759,633, MDA 19, SER 601, BML-190, N-alkylamide, rutamarin, diindolylmethane (DIM) and combinations thereof.

Beta-caryophyllene (trans-(1R,9S)-8-methylene4,11,11 trimethylbicyclo[7.2.0)]undec-4-ene, BCP, CAS 87-44-5) is a CB2-receptor selective agonist (Gertsch et al. 2008, Anavi-Goffer et al., 2012). BCP exhibits chirality at positions 1 and 9 and is the 1R,9S enantiomer, the (−) form.

HU-308 ([(1R,2R,5R)-2-[2,6-dimethoxy-4-(2-methyloctan-2-yl)phenyl]-7, 7-dimethyl-4-bicyclo[3.1.1]hept-3-enyl] methanol) is a synthetic cannabinoid, which is highly selective for the CB2 receptor.

The fact that orally-administered BCP is absorbed by the digestive tract and becomes systemically available and its apparent substantial non-toxicity makes BCP attractive as a potential active pharmaceutical ingredient.

However, BCP whose main commercial use is as food additive, is not commercially available in pharmaceutical grade. The food additive grade contains a relatively low percentage of BCP, contains impurities like BCP oxide, alpha-humulene and BCP (+) enantiomer and is not well defined analytically.

According to Chicca A. et al (Chem. Biol. 2014, 9, 1499-1507), BCP-oxide and alpha-humulene's inactivity suggests the existence of a specific sesquiterpene pharmacophore for CB2 receptor binding in BCP only but not in BCP-oxide and alpha-humulene.

The BCP impurities can have potential negative side-effects on the therapeutic effect of the compositions of this invention.

For example, alpha-humulene is a skin, eyes and respiratory irritant, according to its MSDS. Also, BCP oxide was found to be an allergen (Sköld M, Karlberg A T, Matura M, Börje A, Food Chem. Toxicol. 2006 April; 44(4): 538-45).

In an embodiment, the compositions of this invention use BCP (and/or other CB2 modulators) in substantially pure form, being substantially free of BCP oxide and alpha-humulene.

Chaves (Chaves J S, Planta Med. 2008 November; 74(14):1678-83) reported that alpha-humulene exhibited a rapid onset and relatively good absorption following oral and topical administration. These findings further contribute to an explanation of the topical and systemic anti-inflammatory and antinociceptive properties previously reported for the essential oil and for alpha-humulene obtained from Cordia verbenacea. Humulene is irritant, but only in high doses.

It is documented that BCP has a potentiating effect on humulene. Thus, Legault (J. Pharm. Pharmacol. 2007 December; 59(12): 1643-7) reports an enhancement of the anticancer effect of humulene by BCP.

It is therefore interesting to determine the activity of BCP/alpha-humulene combinations. The experimental data (see examples) suggests that BCP/alpha-humulene combinations are therapeutically active.

Aspects of the invention relate to compositions comprising a combination of BCP and alpha-humulene, optionally with traces of other ingredients like BCP-oxide. In some embodiments, there are provided compositions comprising from about 85% w/w to about 99% w/w BCP and from about 1% w/w to about 15% w/w humulene, with traces of other ingredients like BCP-oxide. In some embodiments, there are provided compositions comprising from about 85% w/w to about 99% w/w BCP and from about 1% w/w to about 15% w/w humulene.

In some embodiments, there is provided a method of treatment of a mental disorder in a patient in need thereof, by administration of a composition comprising from about 85% w/w to about 99% w/w BCP and from 1% w/w to 15% w/w humulene, a self-emulsifying vehicle. In some embodiments, there is provided a method of treatment of a mental disorder in a patient in need thereof, by administration of a composition comprising from about 85% w/w to about 99% w/w BCP and from 1% w/w to 15% w/w humulene, a self-emulsifying vehicle and optionally one or more of the following: a therapeutically effective amount of either at least one antipsychotic agent, at least one GPR55 modulator, at least one anti-inflammatory agent, at least one enzyme enhancer, at least on enzyme inhibitor, at least one antidepressant, at least one anxiolytic, at least one terpene or terpenoid, at least one anti-diabetic agent, at least cognitive enhancer agent or any combinations of the foregoing. In some embodiments, the composition can comprise from about 85% w/w to about 99% w/w BCP and from about 1% w/w to about 15% w/w humulene, with traces of other ingredients like BCP-oxide.

One of the drawbacks of BCP is its proneness to autoxidation. Beta-caryophyllene starts to oxidize immediately when air exposed and after 5 weeks almost 50% of the original compound is consumed. Caryophyllene oxide was found to be the major oxidation product ((Sköld M, Karlberg A T, Matura M, Börje A, Food Chem Toxicol. 2006 April ;44(4):538-45)). The practical effect of this instability is that conventional compositions containing the compounds have relatively short shelf lives, thus making commercial distribution and storage difficult.

In order to maintain the purity, stability and the therapeutic activity, the compositions of this invention comprising BCP and/or other CB2 receptor selective agonists are stabilized by addition of an antioxidant and/or free-radical scavenger. As used herein, term “stable” means that the quantitative composition does not significantly change over the time, during the entire shelf-life of the composition, namely for at least 3 months, advantageously for at least 6 months, more advantageously for at least 12 months, even more advantageously for at least 24 months, under standard conditions, in particular at a temperature ranging for 20° C. to 40° C. and a relative humidity ranging for 30% to 75%. In particular, caryophyllene oxide level is less than 5% by weight, based on the total weight on the composition, during the entire shelf life of the composition. In the present invention, the composition is advantageously stable during 6 months to 1 year or during 1 year to 2 years under standard conditions. In some embodiments, compositions comprising BCP and/or other CB2 receptor selective agonists and further comprising an antioxidant, a free-radical scavenger or a combination of an antioxidant and a free-radical scavenger have an extended shelf-life. In some embodiments, the stable or stabilized compositions have the property to loose less than about 5% of the original compound when stored at room temperature from about one year to about two years. In some embodiments, the stable or stabilized compositions have the property to loose less than about 10% of the original compound when stored at room temperature from about one year to about two years. In some embodiments, the stable or stabilized compositions have the property to loose less than about 4% of the original compound when stored at room temperature from about one year to about two years. In some embodiments, the stable or stabilized compositions have the property to loose less than about 3% of the original compound when stored at room temperature from about one year to about two years. In some embodiments, the stable or stabilized compositions have the property to loose less than about 2% of the original compound when stored at room temperature from about one year to about two years. In some embodiments, the stable or stabilized compositions have the property to loose less than about 1% of the original compound when stored at room temperature from about one year to about two years. In some embodiments, the stable or stabilized compositions have the property to loose from about 5% to about 10% of the original compound when stored at room temperature from about one year to about two years. In some embodiments, the stable or stabilized compositions have the property to loose from about 1% to about 5% of the original compound when stored at room temperature from about one year to about two years. One of the problems related to the use of cannabinoids, in general, and CB2 receptor agonists, in particular, is their low bioavailability. Thus, for example, oral THC is only 4% to 12% bioavailable and its absorption is highly variable (McGilveray L J., Pain Res Manag. 2005 Autumn; 10 Suppl A:15A-22A). The same is true for the oral bioavailability of BCP, a CB2 selective agonist (U.S. Patent Application 2015/0051299 and PCT Application 2013/140342, which are incorporated herein in their entireties).

It should be appreciated that the reasons responsible for low bioavailability via oral route can be due to poor aqueous solubility and/or poor chemical stability in the alkaline pH of the gastro-intestinal tract.

This is why much effort has been invested in the improvement of the cannabinoids' oral bioavailability.

For example, current medications using CBD request high CBD amounts per patient. Echo Pharmaceuticals Ltd has developed a drug delivery technology Alitra™ in which the drug is formulated in a solid composition (granulates) to improve absorption and distribution of compounds with low water solubility. For example, a drug based on CBD was developed (Arvisol®) with 30% bioavailability improvement.

Similarly, among the main disadvantages of currently available Δ⁹-tetrahydrocannabinol (THC) formulations are dosing difficulties due to poor pharmacokinetic characteristics. Namisol® is a novel THC Alitra™ formulation, designed to improve THC absorption (Klumpers L. E., Br J Clin Pharmacol. 2012 July; 74(1): 42-53.). No such research has been carried out on CB2 receptor modulators, or CB2 receptor selective agonists in general or on BCP and liquid formulations in particular.

The composition of this disclosure is based on a formulation of the self-emulsifying drug delivery system (SEDDS) type. The SEDDS technology is based on isotropic mixtures of oils, surfactants, solvents and co-solvents/surfactants, which form fine relatively stable oil-in-water (o/w) emulsions upon aqueous dilution owing to the gentle agitation of the gastrointestinal fluids. In this case, there is no granulate, but rather liquid compositions which can be orally administered in soft or hard gelatin capsules.

A large number of composition alternatives have been explored (see Examples 1-11) in order to develop the most suitable composition for oral delivery of CB2 receptor agonists in general and BCP in particular. Example 1, for example, shows a SEEDS composition that is efficient for oral administration.

A liquid composition for oral delivery is described in Example 12.

Studies have been carried out with water-diluted. SEDDS compositions on mice (see Example 13). In some embodiments, the composition is stabilized by addition to the composition of an antioxidant and/or free-radical scavenger. The stabilization of the composition can be necessary because of the proneness of the CB2 receptor modulators and CB2 receptor agonists to oxidation and can be achieved by addition to the composition of an antioxidant or free-radical scavenger. Antioxidant or free-radical can also potentiate the therapeutic effect of CB2 receptor modulators and CB2 receptor agonists.

In some embodiments, there is provided a composition formulated as a stable self-emulsifying drug delivery system (SEDDS) comprising at least one oil, at least one surfactant HLB<9, at least one surfactant HLB>13, at least one co-surfactant, at least one antioxidant and/or free-radical scavenger, at least one CB2 receptor selective or highly selective agonist and optionally at least one antipsychotic agent. In some embodiments, at least one CB2 receptor selective or highly selective agonist is in a substantially pure form.

In some embodiments, the oil is selected from the group consisting of medium chain triglycerides, propylene glycol dicaprilate/dicaprate, medium chain mono- and diglycerides, acetylated mono- and diglycerides and olive oil and combinations thereof.

In some embodiments, the surfactant HLB<9 is selected from the group consisting of oleoyl polyoxyl-6 glycerides, linoleyl polyoxyl-6 glycerides (20-40%), Polysorbate 85 (Tween-85) polyoxyethylene (20) sorbitan trioleate (5-15%), Span-80 (sorbitan monooleate) (5-25%), polyglyceryl-3 dioleate (15-35%) and glycerin monolinoleate (10-35%), and combinations thereof.

In some embodiments, the surfactant HLB>13 is selected from the group consisting of polyoxylated castor oil (5-25%), PEG 40 hydrogenated castor oil, PEG-15 hydroxystearate (5-25%) and caprylocaproyl polyoxyl-8 glycerides (10-20%), PEG-20 sorbitan monostearate, PEG-20 sorbitan monooleate (5-25%) and PEG 40 stearate (5-25%) and combinations thereof.

In some embodiments, the co-surfactant is selected from the group consisting of soy lecithin (>=75% phosphatidylcholine in oil 1-10% w/w), soy lecithin PC content >50% (2-15%), egg lecithin E-60 or E-80 (1-5%) and distearoylphosphatidylcholine (0.5-3%), and combinations thereof.

In some embodiments, there is provided a composition formulated as a stable self-emulsifying drug delivery system (SEDDS) comprising:

from about 10% w/w to about 50% w/w of an oil selected from the group consisting of medium chain triglycerides, propylene glycol dicaprilate/dicaprate, medium chain mono- and diglycerides, acetylated mono- and diglycerides and olive oil and combinations thereof;

from about 20% w/w to about 50% w/w of a surfactant HLB<9 selected from the group consisting of oleoyl polyoxyl-6 glycerides, linoleyl polyoxyl-6 glycerides (20-40%), Polysorbate 85 (Tween-85) polyoxyethylene (20) sorbitan trioleate (5-15%), Span-80 (sorbitan monooleate) (5-25%), polyglyceryl-3 dioleate (15-35%) and glycerin monolinoleate (10-35%), and combinations thereof;

from about 5% w/w to about 10% w/w of a surfactant HLB>13 selected from the group consisting of polyoxylated castor oil (5-25%), PEG 40 hydrogenated castor oil, PEG-15 hydxoxystearate (5-25%) and caprylocaproyl polyoxyl-8 glycerides (10-20%), and combinations thereof;

from about 5% w/w to about 25 w/w of a surfactant HLB>13 selected from the group consisting of PEG-20 sorbitan monostearate, PEG-20 sorbitan monooleate (5-25%) and PEG 40 stearate (5-25%) and combinations thereof;

from about 0.5% w/w to about 15% w/w of a co-Surfactant selected from the group consisting of soy lecithin: (>=75% phosphatidylcholine in oil, 1-10% w/w), soy lecithin PC content >50% (2-15%), egg lecithin E-60 or E-80 (1-5%) and distearoylphosphatidylcholine (0.5-3%);

from about 0.1% w/w to about 5% w/w of an antioxidant and/or or free radical scavenger selected from the group consisting of d-alpha-tocopherol (1-4% w/w), dl-alpha-tocopherol (25% w/w), dl-alpha-tocopheryl acetate (2-5%), mixed tocopherols (alpha, beta, gama—1-4% w/w), d-alpha-tocopheryl acetate (2-5%), butylated hydroxyanisole (BHA, 0.1-0.5%) and combinations thereof, and combinations thereof;

from about 1% w/w to about 20% w/w of at least one CB2 receptor agonist in substantially pure form; and

optionally from about 0.1% w/w to about 5% w/w of at least one antipsychotic agent.

In some embodiments, there is provided a composition formulated as a stable self-emulsifying drug delivery system (SEDDS) comprising:

from about 30% w/w to about 50% w/w capric/caprylic triglycerides;

from about 30% w/w to about 50% w/w oleoyl polyoxyl-6 glycerides;

from about 5% w/w to about 10% w/w polyoxylated castor oil;

from about 7% w/w to about 15% w/w PEG-20 sorbitan monostearate;

from about 2% w/w to about 5% w/w soy lecithin (75% phosphatidylcholine in oil);

from about 1% w/w to about 3% w/w d-alpha tocopherol;

from about 1% w/w to about 20% w/w of at least one CB2 receptor agonist in substantially pure form; and

optionally from about 0.1% w/w to about 5% w/w of at least one antipsychotic agent.

In some embodiments, the at least one CB2 receptor agonist in the composition of this disclosure is selected from the group consisting of BCP, HU-308, HU-433, HU-910, HU-914, CB 65, GP 1a, GP 2a, GW 405833, JWH 015, JWH 133, AM1241, L-759,656, L-759,633, MDA 19, SER 601, BML-190, N-alkylamide, rutamarin, diindolylmethane (DIM) and combinations thereof.

In some embodiments, the at least one agent in the composition of this disclosure is selected from the group consisting of an antipsychotic agent, a GPR55 modulator, an anti-inflammatory agent, an enzyme enhancer, an enzyme inhibitor, an antidepressant, an anxiolytic, a terpene or terpenoid, an anti-diabetic agent, a cognitive enhancer agent and combinations thereof.

In some embodiments, the at least one agent in the composition of this disclosure is selected from the group consisting of a limonene, pinene, linalool, myracene, thujone, polypeptide-p, rosmarinic acid, charantin, methylhydroxy chalcone polymer, coumarin, curcumine, piperine, CB1 receptor antagonists and combinations thereof.

In some embodiments, the at least one agent in the composition of this disclosure is selected from the group consisting of the group of modulators that targeting the enzymes cyclooxygenase-2 (COX-2), fatty acid amide hydrolase (FAAH), monoacylglycerol lipase (MGL), α/β-hydrolase domain containing 6 (ABDH6 or ABHD6), α/β-hydrolase domain containing 12 (ABDH12), α/β-hydrolase domain containing 4 (ABDH4), sn-1-diacylglycerol lipase alpha (DAGLalpha), sn-1-diacylglycerol lipase beta (DAGLbeta), N-acyl phosphatidylethanolamine phospholipase D (NAPE-PLD), phosphodiesterase 1 (GDE1), phospholipase C (PLC), phospholipase D (PLD) and combination thereof.

In some embodiments, the at least one antipsychotic agent in the composition of this disclosure is selected from the group consisting of benperidol, bromperidol, droperidol, haloperidol, timiperone, fluspirilene, penfluridol, pimozide, acepromazine, chlorpromazine, cyamemazine, dixyrazine, fluphenazine, levomepromazine, mesoridazine, perazine, pericyazine, perphenazine, pipotiazine, prochlorperazine, promazine, promethazine, prothipendyl, thioproperazine, thioridazine, trifluoperazine, triflupromazine, chlorprothixene, clopenthixol, flupentixol, thiothixene, zuclopenthixol, amisulpride, amoxapine, aripiprazole, dehydroaripiprazole, asenapine, cariprazine, clozapine, blonanserin, iloperidone, lurasidone, melperone, nemonapride, olanzapine, paliperidone, paliperidone palmitate, perospirone, quetiapine, remoxipride, risperidone, sertindole, sultopride, trimipramine, ziprasidone, brexpiprazole, ITI-007, pimavanserin, RP5063 (RP5000) cannabidiol (CBD), cannabidivarin (CBDV), cannabiodiolic acid (CBDA), tetrahydrocannabivarin (THCV), OPC-14857, DM-1458, DM-1451, DM-1452, DM-1454, DCPP, cannabigerol (CBG) and its analogs CBGA and CBGV and combinations thereof.

In some embodiments, the at least one antipsychotic agent may belong to several types or subclasses.

In some embodiments, the composition described herein further comprises, in addition to a CB2 selective receptor agonist, at least one antipsychotic agent, such as, for example, a typical antipsychotic agent including, but not limited to, one or more of a butyrophenone type antipsychotic agent selected from the group consisting of haloperidol, droperidol, benperidol, trifluperidol, melperone, lenperone, azaperone, domperidone, butyrophenone, fluanisone, penfluridol, pipamperone, spiperone, nonaperone, bromperidol and timiperone, a diphenylbutylpiperidine type antipsychotic agent selected from the group consisting of luspirilene, penfluridol, pimozide, clopimozide, fluspirilene, penfluridol, a phenothiazine type antipsychotic acid agent selected from the group consisting of acepromazine, chlorpromazine, cyamemazine, dixyrazine, fluphenazine, levomepromazine, mesoridazine, perazine, pericyazine, perphenazine, pipotiazine, prochlorperazine, promazine, promethazine, prothipendyl, thioproperazine, thioridazine and trifluoperazine and triflupromazine, a thioxanthene type antipsychotic agent selected from the group consisting of chlorprothixene, clopenthixol, flupentixol, thiothixene and zuclopenthixol and/or an atypical antipsychotic agent including, but not limited to one or more of an atypical antipsychotic agent usually belonging to the D2 antagonist/inverse agonist, 5-HT2A antagonist/inverse agonist types selected, from the group consisting of amisulpride, amoxapine, asenapine, cariprazine, clozapine, blonanserin, iloperidone, lurasidone, melperone, nemonapride, olanzapine, paliperidone, paliperidone palmitate, perospirone, quetiapine, remoxipride, risperidone, sertindole, sultopride, trimipramine, ziprasidone, ITI-007, pimavanserin (ACP-103; 5-HT2A antagonist), and combinations thereof, and/or an atypical antipsychotic agent including, but not limited to one or more of an atypical antipsychotic agent usually belonging to the D2 partial agonist types selected from the group consisting aripiprazole and its metabolites OPC-14857, DM-1458, DM-1451, DM-1452, DM-1454 and DCPP, brexpiprazole and RP5063 (RP5000) and combinations thereof and/or a cannabinoid exhibiting antipsychotic activity selected from the group consisting of tetrahydrocannabivarin (THCV—CB1 antagonist; CB2 receptor partial agonist), cannabidiol (CBD—CB1/CB2/GPR55/ABn-CBD antagonist/inhibitor) and cannabigerol (CBG—CB1/CB2 partial agonist).

In some embodiments, there are provided compositions comprising combinations of a CB2 selective receptor agonist from one of the above types or subclasses with an antipsychotic agent from one of the above types or subclasses.

In some embodiments, there is provided a composition wherein the at least one CB2 receptor agonist is beta-caryophyllene (BCP) as sole active agent.

According to some embodiments, there is provided a composition wherein the at least one CB2 receptor agonist is beta-caryophyllene (BCP) in a mixture with humulene and traces of BCP oxide.

In some embodiments, there is provided a composition wherein the at least one CB2 receptor agonist is beta-caryophyllene (BCP) and the at least one antipsychotic agent is selected from the group consisting of risperidone, paliperidone, paliperidone palmitate, aripiprazole, quetiapine, CBD and its analogs, THCV, brexpiprazole and combinations thereof.

In certain embodiments, the composition of this disclosure can be formulated for oral, topical, intranasal or rectal administration.

In other embodiments, the composition of this disclosure is formulated for oral administration, wherein in the form of a capsule, suspension, liquid composition for oral administration, solution, emulsion or syrup.

In another embodiment, the topical composition of this disclosure is formulated as a transdermal gel, cream, patch or topical spray.

The role of CB2 receptor selective agonists, in general, and BCP, in particular, in the treatment of schizophrenia, has not previously been studied.

The effect of BCP in various compositions and modes of administration in a murine model of schizophrenia, produced by administration of the N-methyl-D-aspartic acid (NMDA) antagonist, phenylcyclidine (PCP) has been described in U.S. Patent Application 2015/0051299 and PCT Application 2013/140342 (incorporated herein in their entireties; description of EXAMPLE 15II. “Postnatal induction of schizophrenia (days 3-15) followed by treatment of adolescent mice with BCP (postnatal days 43-61) and FIGS. 14A-14E show results demonstrating that BCP treatment at adolescence reversed the effect of PCP on ambulation but did not affect body weight; line graph of body weight at PHD 40-68 (14A), bar graph of female and male body weight at PMD63 (14B), line graph of male ambulation at PND 63 (14D), line graph of female ambulation at PND 63 and line graph of male and female ambulation at PHD 63”).

According to some aspects, there is provided a composition comprising beta-caryophyllene (BCP) and a self-emulsifying vehicle for use in treating schizophrenia.

According to some aspects, there is also provided the use of beta-caryophyllene (BCP) and a self-emulsifying vehicle in the manufacture of a medicament for treating schizophrenia in a subject in need thereof.

In some embodiments, such a composition is formulated for administration to a human subject. In some embodiments, such a composition is formulated for administration to a non-human animal subject.

According to some aspects of the invention, there is also provided a method for treating schizophrenia in a subject in need thereof, the method comprising administering a pharmaceutically-effective amount of beta-caryophyllene (BCP) to a subject in need thereof. In some embodiments, the subject is a human subject. In some embodiments, the subject is a non-human animal.

The efficacy of the methods and compositions according to the teachings herein are demonstrated in the experimental: section herein below.

According to some embodiments, the compositions and methods of treatments disclosed herein are useful for treating one or more of paranoid schizophrenia, disorganized schizophrenia, undifferentiated schizophrenia, catatonic schizophrenia, and residual schizophrenia.

In some embodiments, the compositions and methods of treatments disclosed herein are useful in the treatment of a negative symptom of schizophrenia (according to The Diagnostic and Statistical Manual of Mental Disorders (DSM)). In some embodiments, the compositions and methods of treatments disclosed herein are useful in the treatment of a positive symptom of schizophrenia.

In some embodiments, the compositions and methods of treatments disclosed herein are useful in the treatment of another symptom of schizophrenia (e.g. cognitive symptoms).

The duration of treatment according to the method of treating schizophrenia according to aspects of the invention is any suitable duration as determined by a treating health-care professional, typically a psychiatric doctor.

The CB2 receptor agonist (or specifically BCP) regimen of administration and the unit dosage administered to a mental disorder patient in need thereof can depend on the mode of administration, the efficiency of the composition and the mental disorder to be treated.

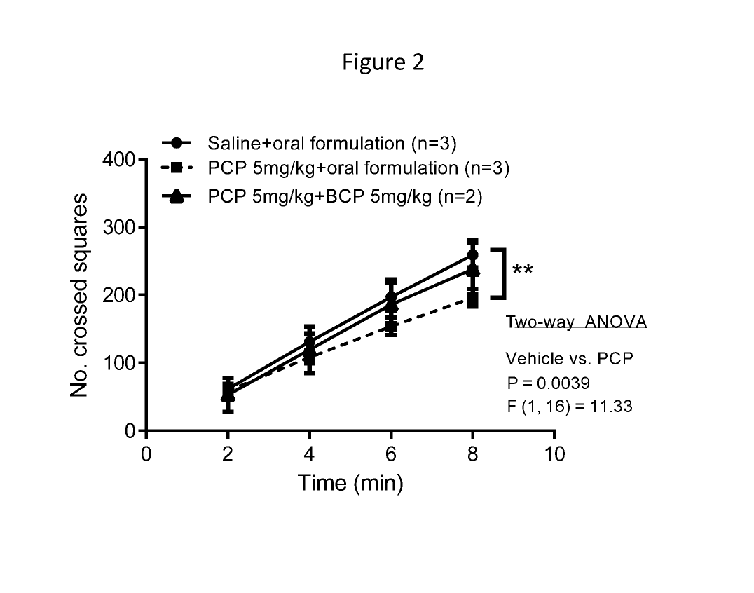

Thus, for example, injectable, nasal and transdermal compositions tend to need lower dosages than some oral compositions. Also, some oral compositions (like the self-emulsifying composition detailed in Example 1 and Example 22) surprisingly require dosages comparable or lower to intraperitoneal injectable compositions (for example, see comparison between the effects of BCP in the open field test after intraperitoneal injection vs. gavage administration of self-emulsifying composition in Example 1). The results of intraperitoneal injection are described in Example 15II and FIG. 14E in U.S. Patent Application 2015/0051299 and PCT Application 2013/140342 to be compared with the results (in FIG. 2 of this disclosure) of gavage administration, in SEDDS composition as described in Example 1 of this disclosure (Description of EXAMPLE 15II. “Postnatal induction of schizophrenia (days 3-15) followed by treatment of adolescent mice with BCP (postnatal days 43-61) and FIGS. 14A-14E show results demonstrating that BCP treatment at adolescence reversed the effect of PCP on ambulation but not affect body weight: line graph of body weight at PND 40-68 (14A), bar graph of female and male body weight at PND63 (14B), line graph of male ambulation at PND 63 (14D), line graph of female ambulation at PND 63 and line graph of male and female ambulation at PND 63”).

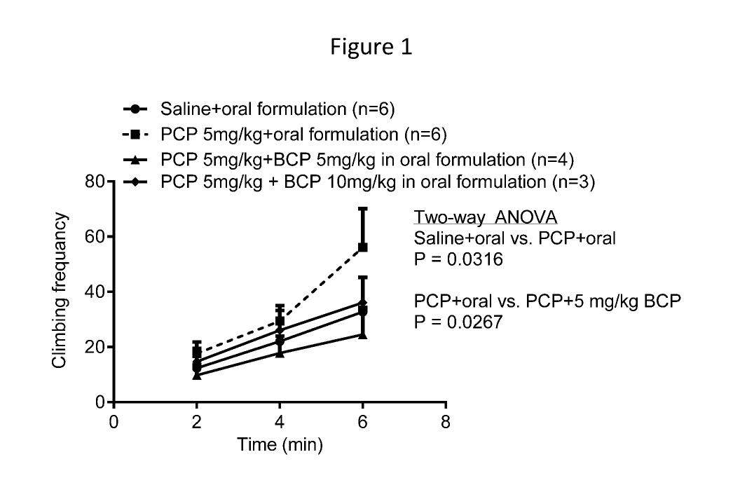

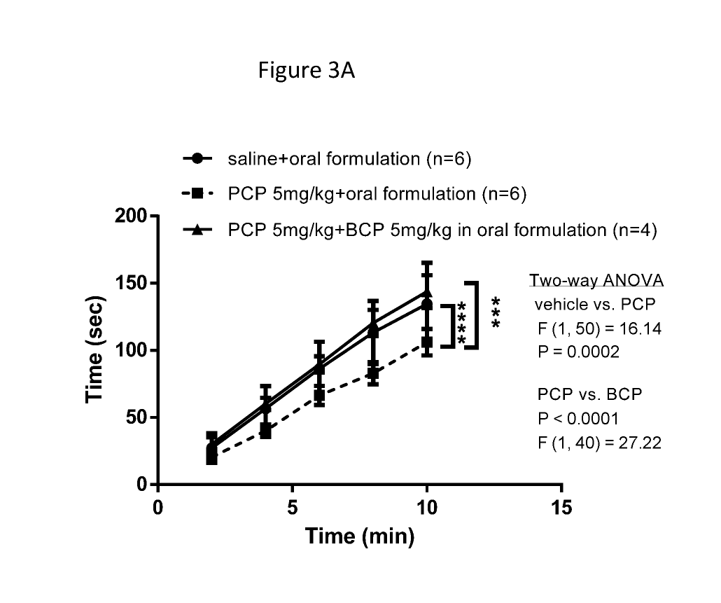

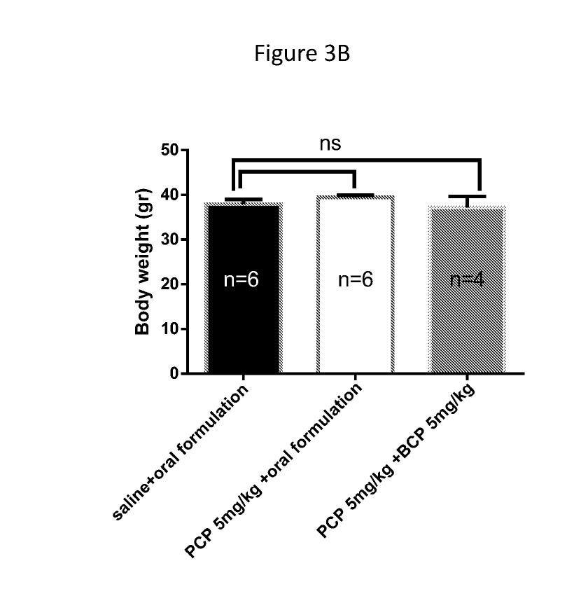

In addition. Example 1 and FIGS. 1 and 3A of this disclosure show that in other tests, i.e. forced-swim test and social interaction test, BCP in self-emulsifying composition is also orally active at about the same dosage (5 mg/kg) as in the open field test (Example 1 and FIG. 2 of this disclosure). Collectively, these results show that surprisingly BCP in SEDDS self-emulsifying composition is orally active at about the same dosage as intraperitoneal injection.

In some embodiments, some SEDDS compositions surprisingly are much more effective than other SEDDS compositions (like in Example 16—V-01 is effective whereas V-02 and V-03are less effective). Collectively, these results show that surprisingly BCP in the specific SEDDS self-emulsifying composition described in Example 1 is orally active at about the same dosage as intraperitoneal injection.

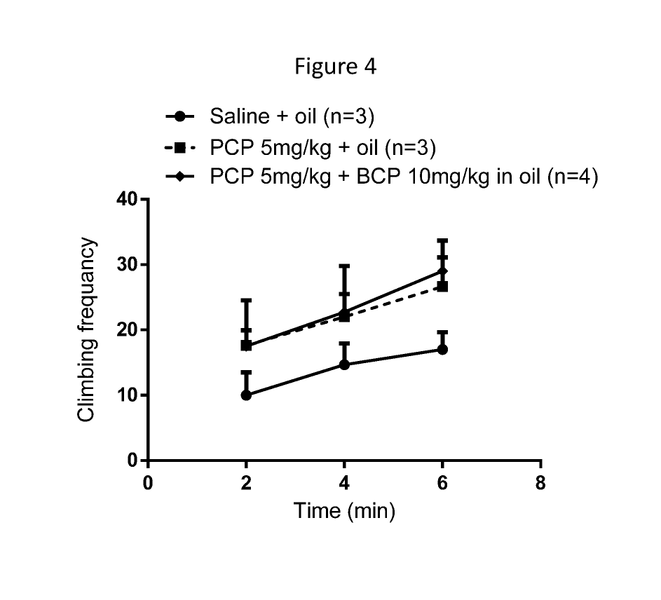

Also, some oral compositions (like the self-emulsifying composition detailed in Example 1) surprisingly are much more effective than other oral compositions (like Example 14—oil composition and compare between the effects of BCP in the forced swim test after gavage administration of self-emulsifying composition in Example 1 and FIG. 1 versus the results after gavage administration of oil composition in Example 14 and FIG. 4 in this application).

In some embodiments, there is provided a highly effective self-emulsifying composition of the present disclosure for the treatment of a mental disease in a patient in need thereof, wherein the administration of an oral dose of said self-emulsifying composition (see Example 1) produces a therapeutic effect similar to the intraperitoneal -administration of the same dose (as above. Example 15II and FIG. 14E in U.S. Patent Application 2015/0051299 and PCT Application 2013/140342) and a much more effective therapeutic effect than non-self-emulsifying oral compositions such as oil compositions (Example 14).

Thus, in some embodiments, the CB2 receptor modulator daily dosage administered to a mental disorder patient in need thereof, by any mode of administration, including but not limited to administration, of slow-release/long-active formulations given on a daily basis, may vary from 0.01 mg/day to 50 mg/day (for highly selective ligands including but not limited, to HU-308) or from 0.1 mg/day to 500 mg/day (for less potent modulators including but not limited to BCP, MH) for highly effective compositions.

In some embodiments, the CB2 receptor modulator daily dosage administered to a mental disorder patient in any mode of administration, including but not limited to administration to a patient in need thereof of slow-release/long-active formulations given on a daily basis, may vary from 0.1 mg/day to 100 mg/day (for highly selective ligands including but not limited to HU-308) or from 1 mg/day to 1000 mg/day (for less potent modulators including but not limited to BCP, MH) for less effective compositions.

Other factors determining the dosage are the age of the patient and effectiveness of the composition. Thus, for BCP for example, a highly effective composition administered daily in any mode of administration, according to some embodiments may be given in an amount of 0.1-10 mg to infants (5-20 kg), 10-20 mg to children (20-50 kg), 20-50 mg to young adults and 50-500 mg to adults (50-100 kg). In some embodiments, for HU-308 for example, a highly effective composition administered daily in any mode of administration may be given in an amount of 0.01-2 mg to in tots (5-20 kg), 2-5 mg to children (20-50 kg), 5-10 mg to young adults and 10-100 mg to adults (50-100 kg). These daily amounts will be administered in one or more discrete dosage units per day or, for highly effective compositions two or three times a week.

In some embodiments, the CB2 receptor modulator, for highly selective ligands including but not limited to HU-308 and for less potent modulators including but not limited to BCP, the daily dosage for less effective compositions may vary from 1 mg/day to 500 mg/day (for highly selective ligands including but not limited to HU-308) or from 10 mg/day to 1000 mg/day (for less potent modulators including but not limited to BCP, MH) for less effective compositions.

In some other embodiments of the method of treating a mental disorder (or specifically schizophrenia or a tic disorder), with a CB2 receptor modulator according to the teachings herein, the average daily amount, in any mode of administration including but not limited to administration in a slow-release/long-active formulations given on a daily basis, for a human subject (especially an adult human, weighing between about 40 kg and about 120 kg) is in the range of from about (for highly potent modulators including but not limited to HU-308) 1 mg to about 25 mg from about 25 mg to about 100 mg, from about 100 mg to about 500 mg such as about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg, about 130 mg, about 150 mg, about 200 mg, about 230 mg, about 350 mg, about 330 mg, about 410 mg, about 460 mg, about 520 mg, about 640 mg, about 770 mg, about 850 mg, about 930 mg, or about 1000 mg (for less potent modulators including but not limited to BCP or MH) or for less effective compositions.

In other embodiments of the method of treating a mental disorder for specifically schizophrenia a tic disorder) according to the teachings herein, the average daily amount of a CB2 receptor modulator in any mode of administration including but not limited to administration in a slow-release/long-active formulations given on a daily basis, for a human subject (especially for an adult human, weighing between about 40 kg and about 120 kg) is in the range of from about 1 mg/day to about 5 mg/day from about 50 mg/day to about 100 mg/day, such as about 5 mg/day, about 10 mg/day, about 30 mg/day, about 50 mg/day, about 70 mg/day from about 100 mg/day for highly selective ligands including but not limited to HU-308, and is in the range of from about 10 mg/day to about 100 mg/day from about 100 mg/day to about 1000 mg/day, such as about 10 mg/day, about 50 mg/day, about 70 mg/day from about 100 mg/day to about 1000 mg/day, such as about 100 mg/day, about 200 mg/day, about 300 mg/day, about 400 mg/day, about 500 mg/day, about 600 mg/day, about 700 mg/day, about 800 mg/day, about 900 mg/day or about 1000 mg/day, for less potent modulators including but not limited to BCP or for less effective compositions. In some embodiments of the method of treating schizophrenia according to the teachings herein, the average daily amount is administered with a frequency of between once per week, twice per week, 3 times pet week, 4 times per week, 5 times per week, 6 times per week, once per day, twice per day, 3 times per day or 4 times per day.

In some embodiments, a composition according to the teachings herein is provided as or made as a dosage form including a plurality of discrete units (e.g., discrete solids or metered liquids, sprays), especially discrete solid units such as pills (including tablets and caplets) and capsules (including gelcaps), wherein each unit includes a CB2 receptor selective modulator or specifically BCP, HU-308 or 4-0-methylhonokiol (MH) in the range of from about 0.05 mg to about 1000 mg, selected from about 0.05 mg, about 0.1 mg, about 0.5 mg, about 1 mg, about 5 mg, about 10 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 200 mg, about 300 mg, about 400 mg, about 500 mg for highly selective ligands including but not limited to HU-308, and in the range of from about 0.1 mg, about 0.5 mg, about 1 mg, about 5 mg, about 10 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 200 mg, about 300 mg, about 400 mg, about 500, about 600 mg, about 700 mg, about 800 mg, about 900 mg, or about 1000 mg for less potent modulators including but not limited to BCP or for less effective compositions. In some such embodiments, such a dosage form is useful for the once-daily administration of the desired average daily dosage, according to age of the patient.

In some embodiments, the dosage of the CB2 receptor modulator administered to a mental disorder patient for highly effective delayed-release delivery compositions (such as compositions for a slow-release, slow-acting form of medication prepared as a capsule or as a depot injection given for example but not limited to intramuscular injection, which are administrated every 1 week or once a month to up to every six months) may vary from 100 mg/single administration (for highly potent modulators including but not limited to HU-308 or for weekly injection) to 3000 mg/single administration (for less potent modulators including but not limited to BCP, MH or for injection every 3 months).

Other factors determining the dosage are the age of the patient and the effectiveness of the composition. Thus, according to some embodiments, for BCP or MH for example, a delayed-release delivery compositions administrated by injection may be given at 0.5-10 mg to infants (5-20 kg), 10-20 mg to children (20-50 kg) and from 100-200 mg to 200-3000 mg to adults (50-100 kg. In some embodiments, for HU-308, for example, a delayed-release delivery compositions administered by injection should be given at 0.5-10 mg to infants (5-20 kg), 10-20 mg to children (20-50 kg) and from 20-100 mg to 100-1000 mg to adults (50-100 kg).

In some embodiments, the CB2 receptor modulator dosage for delayed-release delivery compositions (such as compositions for a slow-release, slow-acting form of medication prepared as a capsule or a depot injection given for example but not limited by intramuscular injection, which are administrated every 1 week, once a month and to up to every three months) may vary from 1 mg/single administration to 500 mg/single administration for less potent modulators including but not limited to BCP or MH, and from 0.1 mg/single administration to 250 mg/single administration for highly potent modulators including but not limited to HU-308.

In some embodiments, the CB2 receptor modulator dosage for delayed release delivery compositions (such as compositions for a slow-release, slow-acting form of medication prepared as a capsule or a depot injection given for example but not limited by intramuscular injection, which are administrated once a month and up to every six months) may vary from 0.5 mg/single administration to 1000 mg/single administration (for highly potent modulators including but not limited, to HU-308) or from 1 mg/single administration to 3000 mg/single administration (for less potent modulators including but not limited to BCP or MH).

Another factor determining the dosage is the effectiveness of the composition. In some embodiments, the dosage for less effective long term delivery compositions in all modes of administration, may vary from 1 mg/day to 3000 mg/day. In some embodiments, the CB2 receptor modulator dosage for delayed release delivery compositions (such as compositions for a slow-release, slow-acting form of medication prepared as a capsule or a depot injection given for example but not limited by intramuscular injection) may vary from 1 mg/single administration to 1000 mg/single administration (for highly potent modulators including but not limited to HU-308) or from 10 mg/single administration to 3000 mg/single administration (for less potent modulators including but not limited to BCP or MH).

Another factor determining the dosage is the age of the patient. Thus, for BCP for example, a delayed-release delivery composition for a slow-release, slow-acting form of medication prepared as a capsule or as a depot injection given for example but not limited to intramuscular injection, which are administrated every 1 week, once a month and to up to every six months, according to some embodiments may be given at an amount of 1-50 mg to infants (5-20 kg), 20-100 mg to children (20-50 kg), 50-200 mg to young adults and from 100-3000 mg to adults (50-500 kg). In some embodiments, for HU-308 for example, a delayed-release delivery composition for a slow-release, slow-acting form of medication prepared as a capsule or a depot injection given for example but not limited by intramuscular injection, which are administrated once a week, once a month and to up to once every six months) according to some embodiments may be given at an amount of 0.1-10 mg to infants (5-20 kg), 5-20 mg to children (20-50 kg) and from 10-100 mg to 50-1000 mg to adults (50-100 kg).

In some embodiments, the administration regimen of delayed-release delivery composition is one administration per week, to once every two weeks, to one administration per a month, to one administration per each other month or once every six months as required.

In some other embodiments of the method of treating schizophrenia according to the teachings herein, the average amount (in mg) per single administration of a delayed-release delivery composition, mainly by injection, (once a week and up to every six months) for a human subject (especially an adult human, weighing between about 40 kg and about 120 kg) is in the range of from about (for highly potent modulators including but not limited to HU-308) 10 mg to about 25 mg from about 25 mg to about 100 mg, from about 100 mg to about 500 mg such as about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg, about 130 mg, about 150 mg, about 200 mg, about 230 mg, about 350 mg, about 330 mg, about 410 mg, about 460 mg, about 500 mg, from about 500 mg to about 1000 mg, such as about 650 mg, about 730 mg, about 840 mg, about 960 mg, about 1000 mg, from about 1000 mg to about 3000 mg, such as about 1200 mg, about 1800 mg, about 2300 mg, about 2500 mg or about 3000 mg (for less potent modulators including but not limited to BCP or MH) or for less effective compositions.

In other embodiments of the method of treating a mental disorder (or specifically schizophrenia) according to the teachings herein, the average amount (in mg) per a single administration of a delayed-release delivery composition mainly by injection (once a week and up to every six months) for a human subject (especially an adult human, weighing between, about 40 kg and about 120 kg) is in the range of from about 10 mg/single administration to about 50 mg/single administration from about 50 mg/single administration to about 100 mg/Single administration, such as about 20 mg/single administration, about 30 mg/single administration, about 60 mg/single administration from about 100 mg/single administration to about 1000 mg/single administration, such as about 200 mg/single administration, about 300 mg/single administration, about 400 mg/single administration, about 500 mg/single administration, about 600 mg/single administration, about 700 mg/single administration, about 800 mg/single administration, about 900 mg/single administration, from about 1000 mg/single administration (for highly potent modulators including but not limited to HU-308) and is in the range of from about 100 mg/single administration to about 3000 mg/single administration, such as about 200 mg/single administration, about 300 mg/single administration, about 400 mg/single administration, about 500 mg/single administration, about 600 mg/single administration, about 700 mg/single administration, about 800 mg/single administration, about 900 mg/single administration, from about 1000 mg/single administration to about 3000 mg/single administration, such as about 1250 mg/single administration about 1600 mg/single administration, about 2100 mg/single administration, about 2400 mg/single administration, about 2700 mg/single administration, or about 3000 mg/single administration (for less potent modulators including but not limited to BCP or MH) or for less effective compositions. In some embodiments of the method of treating schizophrenia according to the teachings herein, the average amount of a single administration mainly, but not limited to injection or oral administration is administered with a frequency of between about once a month to once every two months, to about once every three months, to about once every four months, to about once every-five mouths, to about once every six months.

In some embodiments, a composition according to the teachings herein is provided as or made as a dosage form including a plurality of discrete units (e.g., discrete solids or metered liquids, sprays, depot formulation for injection), especially discrete solid units such as pills (including tablets and caplets) and capsules (including gelcaps), where each unit includes a CB2 receptor selective modulator or specifically BCP, HU-308 and 4-0-methylhonokiol (MH) in the range of from about 10 mg to about 1000 mg, such as about 10 mg, such as about 50 mg, such as about 100 mg, such as about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg for highly selective ligands including but not limited to HU-308, and in the range of from about 100 mg to about 3000 mg, such as about 10 mg, such as about 50 mg, such as about 100 mg, such as about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1500 mg, about 2000 mg, about 2500 mg, or about 3000 mg for less potent modulators including but not limited to BCP or for less effective compositions. In some such embodiments, such a dosage form is useful for a single administration of the desired average dosage per single administration.

According to some embodiments, the compositions of this invention may be administered by any suitable route of administration, including but not limited to oral, parenteral, topical, intranasal, vaginal or rectal administration.

According to some embodiments, there is provided an oral composition formulated as a capsule, suspension, syrup, liquid composition for oral administration, solution, transmucosal lozenge, sachet or sprinkle. The topical composition is formulated as a transdermal gel, cream, patch or topical spray. The intranasal composition is formulated as a nasal spray.

In an embodiment, the composition is a gastro-resistant oral dosage form, that is to say, an orally-administrable dosage form configured to carry the active(s) through the stomach to be released into contact with the digestive tract only after passage through the duodenum. As an example, in some such embodiments, the composition is in the form of a gastro-resistant soft gel capsule, comprising between 5 mg and about 1000 mg BCP in a self-emulsifying vehicle. As an example, in some such embodiments, the composition is in the form of a gastro-resistant soft gel capsule, comprising between 0.5 mg and about 500 mg HU-308 in a self-emulsifying vehicle. Some embodiments of the method, when implemented with an adult human subject, comprise orally ingesting a single such capsule twice a day for at least one a month or once every two months, to about once every three months, to about once every four months, to about once every five months, to about once every six months, so that the average daily amount is between about 10 mg and about 500 mg BCP.

In some embodiments, the composition described herein further comprises at least one antipsychotic agent, such as, for example, a typical antipsychotic agent including, but not limited to, one or more of chlorpromazine, haloperidol, perphenazine, pimozide or fluphenazine, and/or an atypical antipsychotic agent including, but not limited to, one or more of clozapine, risperidone, olanzapine, quetiapine, ziprasidone, aripiprazole, sertindole, amisulpride, paliperidone, paliperidone palmitate, and combinations thereof.

In some embodiments of the method of treatment, the CB2 receptor selective agonist or for example BCP is administered together with at least one antipsychotic agent selected from one or more of a butyrophenone type antipsychotic agent selected from the group consisting of haloperidol, droperidol, benperidol, trifluperidol, melperone, lenperone, azaperone, domperidone, butyrophenone, fluanisone, penfluridol, pipamperone, spiperone, nonaperone, bromperidol and timiperone, a diphenyibutylpiperidine type antipsychotic agent selected from the group consisting of luspirilene, penfluridol, pimozide, clopimozide, fluspirilene, penfluridol, a phenothiazine type antipsychotic acid agent selected from the group consisting of acepromazine, chlorpromazine, cyamemazine, dixyrazine, fluphenazine, levomepromazine, mesoridazine, perazine, pericyazine, perphenazine, pipotiazine, prochlorperazine, promazine, promethazine, prothipendyl, thioproperazine, thioridazine and trifluoperazine and triflupromazine, athioxanthene type antipsychotic agent selected from the group consisting of chlorprothixene, clopenthixol, flupentixol, thiothixene and zuclopenthixol and/or an atypical antipsychotic agent including, but not limited to one or more of an atypical antipsychotic agent usually belonging to the D2 antagonist/inverse agonist, 5-HT2A antagonist/inverse agonist types selected from the group consisting of amisulpride, amoxapine, asenapine, cariprazine, clozapine, blonanserin, iloperidone, lurasidone, melperone, nemonapride, olanzapine, paliperidone, paliperidone palmitate, perospirone, quetiapine, remoxipride, risperidone, sertindole, sultopride, trimipramine, ziprasidone, ITI-007, pimavanserin (ACP-103; 5-HT2A antagonist), and combinations thereof, and/or an atypical antipsychotic agent including, but not limited to one or more of an atypical antipsychotic agent usually belonging to the D2 partial agonist types selected from the group consisting aripiprazole and its metabolites OPC-14857, DM-1458, DM-1451, DM-1452, DM-1454 and DCPP, brexpiprazole and RP5063 (RP5000) and combinations thereof and/or a cannabinoid exhibiting antipsychotic activity selected from the group consisting of tetrahydrocannabivarin (THCV—CB1 antagonist, CB2 receptor partial agonist), cannabidiol (CBD—CB1/CB2/GPR55/ABn-CBD antagonist/inhibitor) and cannabigerol (CBG—CB1/CB2 partial agonist) and combinations thereof.

In some embodiments where the CB2 receptor selective agonist or for example BCP and an antipsychotic agent are administered together, the two active agents can be co-administered in a single dosage form.

In some embodiments where the BCP and an antipsychotic agent are administered together, the CB2 receptor modulator or for example BCP and the antipsychotic agent can be co-administered in separate dosage forms, either sequentially or simultaneously. For example, the additional antipsychotic agent may be administered prior to administration of the CB2, or the additional antipsychotic agent may be administered subsequent to administration of CB2.

While not wishing to be bound to any one theory, the inventor consider that it is likely that at least part, if not all, of the herein demonstrated efficacy of the CB2 receptor modulators or CB2 receptor selective agonists in general or BCP in particular in treating schizophrenia relates to the CB2 receptor selective agonist properties.

According to an aspect of some embodiments of the teachings herein, there is also provided the use of a CB2 receptor selective or highly selective agonist and a self-emulsifying vehicle in the manufacture of a medicament for treating schizophrenia in a subject in need thereof.

According to some aspects, there is also provided a method for treating schizophrenia in a subject in need thereof, the method comprising administering a pharmaceutically-effective amount of a CB2 selective receptor agonist to the subject.

In some embodiments, there is provided, a stable self-emulsifying composition for treatment of mental disorders in a patient in need thereof, comprising a therapeutically effective amount of at least one CB2 receptor modulator in substantially pure form, a self-emulsifying vehicle and optionally a therapeutically effective amount of at least one antipsychotic agent, wherein the at least one CB2 receptor modulator and the at least one antipsychotic agent are substantially solubilized. In this context, “substantially solubilized” means that more than 90% w/w, preferably more than 95% w/w and even more preferably more than 99% w/w are solubilized.

The self-emulsifying composition spontaneously forms an oil-in-water emulsion, typically with an average particle size below 1 micron (see Example 1) upon dilution with water containing media or body fluid. The average particle size of the emulsion depends on the composition comprising the self-emulsifying vehicle and the active agent(s).

In some embodiments, there is provided a self-emulsifying composition for treatment of mental disorders in a patient in need thereof, wherein said composition is physically stable at least 2 hours during the time required for effective absorption in the gastrointestinal tract, and wherein said composition spontaneously forms an oil-in-water emulsion upon dilution with water containing media or body fluid. The GI tract transition time is a function of many factors, like gastric emptying rate and intestinal transit rate, but about 10 hrs GI stability is considered to be sufficient.

The droplet (particle) size of the above emulsion is smaller than 10 mcm, preferably smaller than 1 mcm more preferably smaller than 500 nm, most preferably smaller than 150 nm.

According to some embodiments, the at least one CB2 receptor modulator in the above composition can be selected from the group consisting of at least one CB2 receptor agonist or partial-agonist, at least one CB2 receptor antagonist or inverse agonist, at least one CB2 receptor antagonist or inverse agonist which is also a selective estrogen receptor modulator (SERM), at least one type of CB2 receptor allosteric modulator and combinations thereof.

In some embodiments, the at least one CB2 receptor agonist or partial agonist in the above composition is selected from the group consisting of BCP, HU-308, HU-433, HU-910, HU-914, CB 65, GP 1a, GP 2a, GW 405833, JWH 015, JWH 133, AM1241, L-759,656, L-759,633, MDA 19, SER 601, BML-190, N-alkylamide, rutamarin, diindolylmethane (DIM), cannabinor (PRS211,375), 2-arachidonoylglycerol, anandamide, delta-9-THC, CP55940, W1N55212-2, HU210, analogs thereof, derivatives thereof and combinations thereof.

In some embodiments, the at least one CB2 receptor antagonist or inverse agonist of the above composition is selected from the group consisting of AM630, JTE-907, SR144528, COR170, 4-0-methylhonokiol (MH), GS12021 (4-0-methylhonokiol analog), cannabinol, 01238, 01184, analogs thereof, derivatives thereof and combinations thereof.

In some embodiments, the at least one CB2 receptor allosteric modulator of the above composition is selected from the group consisting of dihydrogambogic acid, garcinolic acid, (-)-5′-dimethylheptyl-cannabidiol (DMH-CBD), analogs thereof, derivatives thereof and combinations thereof.

In some embodiments, the at least one CB2 receptor modulator which is also a selective estrogen receptor modulator (SERM) of the above composition is selected from the group consisting of raloxifene, bazedoxifen, lasofoxifene, tamoxifen, afimoxifene, arzoxifene, ormeloxifene, toremifene, ospemifene, analogs thereof, derivatives thereof and combinations thereof.

In some embodiments, the at least one antipsychotic agent of the above composition is selected from the group consisting of one or more of a butyrophenone type antipsychotic agent selected from the group consisting of haloperidol, droperidol, benperidol, trifluperidol, melperone, lenperone, azaperone, domperidone, butyrophenone, fluanisone, penfluridol, pipamperone, spiperone, nonaperone, bromperidol and timiperone, a diphenylbutylpiperidine type antipsychotic agent selected from the group consisting of luspirilene, penfluridol, pimozide, clopimozide, fluspirilene, penfluridol, a penothiazine type antipsychotic acid agent selected from the group consisting of acepromazine, chlorpromazine, cyamemazine, dixyrazine, fluphenazine, levomepromazine, mesoridazine, perazine, pericyazine, perphenazine, pipotiazine, prochlorperazine, promazine, promethazine, prothipendyl, thioproperazine, thioridazine and trifluoperazine and triflupromazine, a thioxanthene type antipsychotic agent selected from the group consisting of chlorprothixene, clopenthixol, flupentixol, thiothixene and zuclopenthixol and/or an atypical antipsychotic agent including, but not limited to one or more of an atypical antipsychotic agent usually belonging to the D2 antagonist/inverse agonist, 5-HT2A antagonist/inverse agonist types selected from the group consisting of amisulpride, amoxapine, asenapine, cariprazine, clozapine, blonanserin, iloperidone, lurasidone, melperone, nemonapride, olanzapine, paliperidone, paliperidone palmitate, perospirone, quetiapine, remoxipride, risperidone, sertindole, sultopride, trimipramine, ziprasidone, ITI-007, pimavanserin (ACP-103; 5-HT2A antagonist), and combinations thereof, and/or an atypical antipsychotic agent including, but not limited to one or more of an atypical antipsychotic agent usually belonging to the D2partial agonist types selected from the group consisting aripiprazole and its metabolites OPC-14857, DM-1458, DM-1451, DM-1452, DM-1454 and DCPP, brexpiprazole and RP5063 (RP5000) and combinations thereof and/or a cannabinoid exhibiting antipsychotic activity selected from the group consisting of tetrahydrocannabivarin (THCV—CB1 antagonist, CB2 receptor partial agonist), cannabidiol (CBD CB1/CB2/GPR55/ABn-CBD antagonist/inhibitor) and cannabigerol (CBG—CB1/CB2 partial agonist) and combinations thereof.

In some embodiments, the, there is provided a composition formulated as a stable self-emulsifying drug delivery system (SEDDS) comprising at least one oil, at least one surfactant HLB<9, at least one surfactant HLB>13, at least one co-surfactant, at least one antioxidant and/or free-radical scavenger, at least one CB2 receptor modulator and optionally an antipsychotic agent and combinations thereof.

In some embodiments, the, the above composition is formulated as a stable self-emulsifying drug delivery system comprising:

from about 10% w/w to about 50% w/w of an oil selected from the group consisting of medium chain triglycerides, propylene glycol dicaprilate/dicaprate, medium chain mono- and diglycerides, acetylated mono- and diglycerides and olive oil and combinations thereof,

from about 20% w/w to about 50% w/w of a surfactant HLB<9 selected from the group consisting of oleoyl polyoxyl-6 glycerides, linoleyl polyoxyl-6 glycerides (20-40%), Polysorbate 85 (Tween-85) polyoxyethylene (20) sorbitan trioleate (5-15%),

Span-80 (sorbitan monooleate) (5-25%), polyglyceryl-3 dioleate (15-35%) and glycerin monolinoleate (10-35%),

from about 5% w/w to about 10% w/w of a surfactant HLB>13 selected from the group consisting of polyoxylated castor oil (5-25%), PEG 40 hydrogenated castor oil, PEG-15 hydroxystearate (5-25%) and caprylocaproyl polyoxyl-8 glycerides (10-20%)

from about 5% w/w to about 25% -w/w of a surfactant HLB>13 selected from the group consisting of PEG-20 sorbitan monostearate, PEG-20 sorbitan monooleate (5-25%)-and PEG 40 stearate (5-25%),

from about 0.5% w/w to about 15% w/w of a co-surfactant selected from the group consisting of soy lecithin (>=75% phosphatidylcholine in oil, 1-10% w/w), soy lecithin PC content >50% (2-15%), egg lecithin E-60 or E-80 (1-5%) and distearoylphosphatidylcholine (0.5-3%),

from about 0.1% w/w to about 5% w/w of aa antioxidant or free radical scavenger selected from the group consisting of d-alpha-tocopherol (1-4% w/w), dl-alpha-tocopherol (25% w/w), dl-alpha-tocopheryl acetate (2-5%), mixed tocopherols (alpha, beta, gama—1-4% w/w), d-alpha-tocopheryl acetate (2-5%), butylated hydroxyanisole (BHA, 0.1-0.5%) and combinations thereof,

from about 1% w/w to about 20% w/w of at least one CB2 receptor modulator in substantially pure form and optionally

from about 0.1% w/w to about 5% w/w of at least one antipsychotic agent

In some embodiments, the above composition is formulated as a stable self-emulsifying drug delivery system (SEDDS) comprising:

from about 30% w/w to about 50% w/w capric/caprylic triglycerides

from about 30% w/w to about 50% w/w oleoyl polyoxyl-6 glycerides4

from about 5% w/w to about 10% w/w polyoxylated castor oil

from about 7% w/w to about 15% w/w PEG-20 sorbitan monostearate

from about 2% w/w to about 5 w/w soy lecithin (75% phosphatidylcholine in oil)

from about 1% w/w to about 3% w/w d-alpha tocopherol

from about 1% w/w to about 20% w/w of at least one CB2 receptor modulator in substantially pure form and optionally

from about 0.1% w/w to about 5% w/w of at least one antipsychotic agent

In some embodiments, the at least one CB2 receptor agonist in the above composition is beta-caryophyllene (BCP) as sole active agent.

In some embodiments, the at least one CB2 receptor agonist in the above composition is beta-caryophyllene (BCP) and the at least one antipsychotic agent is selected from the group consisting of risperidone, paliperidone, paliperidone palmitate, aripiprazole, quetiapine, CBD and its analogs, THCV, brexpiprazole and combinations thereof.

In a further embodiment, in the above composition, the at least one CB2 receptor agonist is beta-caryophyllene (BCP) and the at least one antipsychotic agent is selected from the group consisting of an of extract of cannabis species comprising 10-98% CBD and its analogs and/or 10-98% THCV and its analogs and/or 10-98% CBG and its analogs and combinations thereof.

In some embodiments, the at least one CB2 receptor agonist in the above composition is [(1R,2R, 5R)-2-[2,6-dimethoxy-4-(2-methyloctan-2-yl) phenyl]-7,7-dimethyl-4-bicyclo[3.1.1]hept-3-enyl] methanol (HU-308) as sole active agent.

In another embodiment, the at least one CB2 receptor agonist in the above composition is HU-308 and the at least one antipsychotic agent is selected from the group consisting of risperidone, paliperidone, paliperidone palmitate, aripiprazole, quetiapine, CBD and its analogs, THCV, brexpiprazole and combinations thereof.

According to an embodiment, the composition of the instant invention is stabilized by addition of an antioxidant or a free-radical scavenger.

In some embodiments, the ratio of antioxidant/CB2 modulator, such as but not-limited to BCP, is from 1:1 to 2:1 w/w. In some embodiments, the antioxidant/CB2 modulator ratio is from 1:1 to 3:1 w/w. In some embodiments, the ratio of antioxidant/CB2 modulator is from 1:1 to 4:1 w/w. In some embodiments, the ratio of antioxidant/CB2 modulator is from 1:1 to 5:1 w/w. In some embodiments, the ratio of antioxidant/CB2 modulator is from 2:1 to 3:1 w/w. In some embodiments, the ratio of antioxidant/CB2 modulator is from 2:1 to 4:1 w/w. In some embodiments, the ratio of antioxidant/CB2 modulator is from 2:1 to 5:1 w/w. In some embodiments, the ratio of antioxidant/CB2 modulator is from 3:1 to 4:1 w/w. In some embodiments, the ratio of antioxidant/CB2 modulator is from 3:1 to 5:1 w/w. In some embodiments, the ratio of antioxidant/CB2 modulator is from 1:1 to 10:1 w/w. In some embodiments, the ratio of antioxidant/CB2 modulator is from 2:1 to 10:1 w/w. In some embodiments, the ratio of antioxidant/CB2 modulator is from 3:1 to 10:1 w/w. In some embodiments, the ratio of antioxidant/CB2 modulator is from 4:1 to 10:1 w/w. In some embodiments, the ratio of antioxidant/CB2 modulator is from 5:1 to 10:1 w/w. In some embodiments, the ratio of antioxidant/CB2 modulator is from 6:1 to 10:1 w/w. In some embodiments, the ratio of antioxidant/CB2 modulator is from 7:1 to 10:1 w/w. In some embodiments, the ratio of antioxidant/CB2 modulator is from 8:1 to 10:1 w/w. In some embodiments, the ratio of antioxidant/CB2 modulator is from 9:1 to 10:1 w/w. In some embodiments, the ratio of antioxidant/CB2 modulator is from 5:1 to 15:1 w/w. In some embodiments, the ratio of antioxidant/CB2 modulator is from 5:1 to 20:1 w/w. In some embodiments, the ratio of antioxidant/CB2 modulator is from 5:1 to 25:1 w/w. In some embodiments, the ratio of antioxidant/CB2 modulator is from 5:1 to 30:1 w/w. In some embodiments, the ratio of antioxidant/CB2 modulator is from 5:1 to 35:1 w/w. In some embodiments, the ratio of antioxidant/CB2 modulator is from 5:1 to 40:1 w/w. In some embodiments, the ratio of antioxidant/CB2 modulator is from 10:1 to 15:1 w/w. In some embodiments, the ratio of antioxidant/CB2 modulator is from 10:1 to 20:1 w/w. In some embodiments, the ratio of antioxidant/CB2 modulator is from 10:1 to 25:1 w/w. In some embodiments, the ratio of antioxidant/CB2 modulator is from 10:1 to 30:1 w/w. In some embodiments, the ratio of antioxidant/CB2 modulator is from 10:1 to 35:1 w/w. In some embodiments, the ratio of antioxidant/CB2 modulator is from 10:1 to 40:1 w/w. In some embodiments, the ratio of antioxidant/CB2 modulator is from 15:1 to 20:1 w/w. In some embodiments, the ratio of antioxidant/CB2 modulator is from 15:1 to 25:1 w/w. In some embodiments, the ratio of antioxidant/CB2 modulator is from 15:1 to 30:1 w/w. In some embodiments, the ratio of antioxidant/CB2 modulator is from 15:1 to 35:1 w/w. In some embodiments, the ratio of antioxidant/CB2 modulator is from 15:1 to 40:1 w/w. In some embodiments, the ratio of antioxidant/CB2 modulator is from 20:1 to 25:1 w/w. In some embodiments, the ratio of antioxidant/CB2 modulator is from 20:1 to 30:1 w/w. In some embodiments, the ratio of antioxidant/CB2 modulator is from 20:1 to 35:1 w/w. In some embodiments, the ratio of antioxidant/CB2 modulator is from 20:1 to 40:1 w/w. In some embodiments, the ratio of antioxidant/CB2 modulator is from 25:1 to 30:1 w/w. In some embodiments, the ratio of antioxidant/CB2 modulator is from 25:1 to 35:1 w/w. In some embodiments, the ratio of antioxidant/CB2 modulator is from 25:1 to 40:1 w/w. In some embodiments, the ratio of antioxidant/CB2 modulator is from 30:1 to 35:1 w/w. In some embodiments, the ratio of antioxidant/CB2 modulator is from 30:1 to 40:1 w/w. In some embodiments, the above composition can spontaneously form an oil-in-water emulsion upon dilution with water containing media or body fluid.

In some embodiments, the ratio of antioxidant/CB2 modulator, such as but not limited to 4-0-methylhonokiol (MH), is from 40:1 to 2500:1 w/w. In some embodiments, the antioxidant/CB2 modulator is from 40:1 to 80:1 w/w. In some embodiments, the ratio of antioxidant/CB2 modulator is from 40:1 to 100:1 w/w. In some embodiments, the ratio of antioxidant/CB2 modulator is from 100:1 to 500:1 w/w. In some embodiments, the ratio of antioxidant/CB2 modulator is from 500:1 to 1000:1 w/w. In some embodiments, the ratio of antioxidant/CB2 modulator is from 1000:1 to 1500:1 w/w. In some embodiments, the ratio of antioxidant/CB2 modulator is from 1500:1 to 2000:1 w/w. In some embodiments, the ratio of antioxidant/CB2 modulator is from 2000:1 to 2500:1 w/w. In some embodiments, the ratio of antioxidant/CB2 modulator is from 3:1 to 5:1 w/w. In some embodiments, the ratio of antioxidant/CB2 modulator is from 40:1 to 100:1 w/w. In some embodiments, the ratio of antioxidant/CB2 modulator is from 40:1 to 50:1 w/w. In some embodiments, the ratio of antioxidant/CB2 modulator is from 40:1 to 60:1 w/w. In some embodiments, the ratio of antioxidant/CB2 modulator is from 40:1 to 80:1 w/w. In some embodiments, the ratio of antioxidant/CB2 modulator is from 60:1 to 500:1 w/w. In some embodiments, the ratio of antioxidant/CB2 modulator is from 80:1 to 500:1 w/w. In some embodiments, the ratio of antioxidant/CB2 modulator is from 100:1 to 500:1 w/w. In some embodiments, the ratio of antioxidant/CB2 modulator is from 150:1 to 250:1 w/w. In some embodiments, the ratio of antioxidant/CB2 modulator is from 150:1 to 280:1 w/w. In some embodiments, the ratio of antioxidant/CB2 modulator is from 150:1 to 300:1 w/w. In some embodiments, the ratio of antioxidant/CB2 modulator is from 200:1 to 500:1 w/w. In some embodiments, the ratio of antioxidant/CB2 modulator is from 300:1 to 500:1 w/w. In some embodiments, the ratio of antioxidant/CB2 modulator is from 400:1 to 500:1 w/w. In some embodiments, the ratio of antioxidant/CB2 modulator is from 600:1 to 1000:1 w/w. In some embodiments, the ratio of antioxidant/CB2 modulator is from 700:1 to 1000:1 w/w. In some embodiments, the ratio of antioxidant/CB2 modulator is from 800:1 to 1000:1 w/w. In some embodiments, the ratio of antioxidant/CB2 modulator is from 900:1 to 1000:1 w/w. In some embodiments, the ratio of antioxidant/CB2 modulator is from 1000:1 to 1200:1 w/w. In some embodiments, the ratio of antioxidant/CB2 modulator is from 1000:1 to 1300:1 w/w. In some embodiments, the ratio of antioxidant/CB2 modulator is from 1000:1 to 1400:1 w/w. In some embodiments, the ratio of antioxidant/CB2 modulator is from 1200:1 to 1400:1 w/w. In some embodiments, the ratio of antioxidant/CB2 modulator is from 1200:1 to 1500:1 w/w. In some embodiments, the ratio of antioxidant/CB2 modulator is from 1300:1 to 1500:1 w/w. In some embodiments, the ratio of antioxidant/CB2 modulator is from 1400:1 to 1500:1 w/w. In some embodiments, the ratio of antioxidant/CB2 modulator is from 1500:1 to 1600:1 w/w. In some embodiments, the ratio of antioxidant/CB2 modulator is from 1500:1 to 1700:1 w/w. In some embodiments, the ratio of antioxidant/CB2 modulator is from 1500:1 to 1800:1 w/w. In some embodiments, the ratio of antioxidant/CB2 modulator is from 1500:1 to 1700:1 w/w. In some embodiments, the ratio of antioxidant/CB2 modulator is from 1500:1 to 1800:1 w/w. In some embodiments, the ratio of antioxidant/CB2 modulator is from 1500:1 to 1900:1 w/w. In some embodiments, the ratio of antioxidant/CB2 modulator is from 1500:1 to 2000:1 w/w. In some embodiments, the ratio of antioxidant/CB2 modulator is from 1600:1 to 2000:1 w/w. In some embodiments, the ratio of antioxidant/CB2 modulator is from 1700:1 to 2000:1 w/w. In some embodiments, the ratio of antioxidant/CB2 modulator is from 1800:1 to 2000:1 w/w. In some embodiments, the ratio of antioxidant/CB2 modulator is from 2000:1 to 2200:1 w/w. In some embodiments, the ratio of antioxidant/CB2 modulator is from 2000:1 to 2300:1 w/w. In some embodiments, the ratio of antioxidant/CB2 modulator is from 2000:1 to 2400:1 w/w. In some embodiments, the ratio of antioxidant/CB2 modulator is from 2000:1 to 2500:1 w/w. In some embodiments, the ratio of antioxidant/CB2 modulator is from 2100:1 to 2500:1 w/w. In some embodiments, the ratio of antioxidant/CB2 modulator is from 2200:1 to 2500:1 w/w. In some embodiments, the ratio of antioxidant/CB2 modulator is from 2300:1 to 2500:1 w/w. In some embodiments, the ratio of antioxidant/CB2 modulator is from 2400:1 to 2500:1 w/w. In some embodiments, the above composition can spontaneously form an oil-in-water emulsion upon dilution with water containing media or body fluid.

The composition of the present disclosure can be formulated for oral, topical, intranasal, vaginal or rectal administration.

The oral composition of this disclosure can be formulated as a capsule, liquid composition for oral delivery, suspension, solution, emulsion or syrup.

The topical composition of this disclosure can be formulated as a transdermal gel, cream, patch or topical spray.

The intranasal composition of this disclosure can be formulated as a nasal spray.

In some embodiments, there is provided a composition of the present disclosure wherein the at least one CB2 receptor modulator is a CB2 selective agonist and is beta caryophyllene (BCP) in substantially pure form as sole active agent and the mental disorder is schizophrenia of all types, onset at any age.

In another embodiment, the at least one CB2 receptor selective agonist in substantially pure form is beta caryophyllene (BCP), the at least one antipsychotic agent is selected from the group consisting of risperidone, paliperidone, paliperidone palmitate, aripiprazole, quetiapine, CBD and its analogs, THCV, brexpiprazole and combinations thereof and the mental disorder is schizophrenia. The BCP in the above composition comprises either one of the two BCP isomers E-BCP and Z-BCP wherein in substantially pure form or mixtures thereof and is substantially free of BCP oxide and a-humulene.

In yet another embodiment, the BCP in the above composition comprises substantially pure isomer E-BCP and is substantially free of BCP oxide and a-humulene.

In a further embodiment, the BCP in the above composition comprises substantially pure isomer Z-BCP and is substantially free of BCP oxide and a-humulene.

According to some embodiments, there is provided a method of treatment of a mental disorder in a patient in need thereof, by administration of a composition comprising a therapeutically effective amount of at least one CB2 receptor modulator in essentially pure form and optionally a therapeutically effective amount of at least one antipsychotic agent in a self-emulsifying vehicle. The at least one CB2 receptor modulator in the above method of treatment is selected from the group consisting of at least one CB2 receptor agonist or partial agonist, at least one CB2 receptor antagonist or inverse agonist, at least one CB2 receptor antagonist or inverse agonist which is also a selective estrogen receptor modulator (SERM), at least one type of CB2 receptor allosteric modulator and combinations thereof.

In some embodiments, the CB2 receptor selective agonist or partial agonist in the above method of treatment is selected from the group comprising BCP, HU-308, HD-433, HU-910, HU-914, CB 65, GP 1a, GP 2a, GW 405833, JWH 015, JWH 133, A1V11241, L-759,656, L759,633, MDA 19, SER 601, BML-190, N-alkylamide, rutamarin, diindolylmethane (DIM) and analogs, derivatives and combinations thereof.

In some embodiments, the at least one antipsychotic agent in the above method of treatment is selected from the group consisting of one or more of a butyrophenone type antipsychotic agent selected from the group consisting of haloperidol, droperidol, benperidol, trifluperidol, melperone, lenperone, azaperone, domperidone, butyrophenone, fluanisone, penfluridol, pipamperone, spiperone, nonaperone, bromperidol and timiperone, a diphenylbutylpiperidine type antipsychotic agent selected from the group consisting of luspirilene, penfluridol, pimozide, clopimozide, fluspirilene, penfluridol, a phenothiazine type antipsychotic acid agent selected from the group consisting of acepromazine, chlorpromazine, cyamemazine, dixyrazine, fluphenazine, levomepromazine, mesoridazine, perazine, pericyazine, perphenazine, pipotiazine, prochlorperazine, promazine, promethazine, prothipendyl, thioproperazine, thioridazine and trifluoperazine and triflupromazine, a thioxanthene type antipsychotic agent selected from the group consisting of chlorprothixene, clopenthixol, flupentixol, thiothixene and zuclopenthixol and/or an atypical antipsychotic agent including, but not limited to one or more of an atypical antipsychotic agent usually belonging to the D2 antagonist/inverse agonist, 5-HT2A antagonist/inverse agonist types selected from the group consisting of amisulpride, amoxapine, asenapine, cariprazine, clozapine, blonanserin, iloperidone, lurasidone, melperone, nemonapride, olanzapine, paliperidone, paliperidone palmitate, perospirone, quetiapine, remoxipride, risperidone, sertindole, sultopride, trimipramine, ziprasidone, ITI-007, pimavanserin (ACP-103; 5-HT2A antagonist), and combinations thereof, and/or an atypical antipsychotic agent including, but not limited to one or more of an atypical antipsychotic agent usually belonging to the D2 partial agonist types selected from the group consisting aripiprazole and its metabolites OPC-14857, DM-1458, DM-1451, DM-1452, DM-1454 and DCPP, brexpiprazole and RP5063 (RP5000) and combinations thereof and/or a cannabinoid exhibiting antipsychotic activity selected from the group consisting of tetrahydrocannabivarin (THCV—CB 1 antagonist, CB2 receptor partial agonist), cannabidiol (CBD—CB1/CB2/GPR55/ABn-CBD antagonist/inhibitor) and cannabigerol (CBG—CB1/CB2 partial agonist) and combinations thereof.

In some embodiments, the mental disorder in the above method of treatment is selected from the group consisting of schizophrenia, schizoaffective disorder, bipolar disorder I and II, unipolar disorder, multiple personality disorder, psychotic disorders, depression, psychotic depression, depressive disorders, major depressive disorder, Tourette's syndrome, tic disorders. epilepsy, anxiety disorders, autistic spectrum disorder, enuresis, addiction, withdrawal symptoms associated with addiction, Asperger syndrome, oppositional defiant disorder, behavioral disturbance, agitation, psychosis/agitation associated with Alzheimer's disease, psychosis associated with Parkinson's disease, personality disorders, borderline personality disorder, avoidant personality disorder, attention-deficit/hyperactive disorder (ADHD, ADD, HD), mania, dementia, anorexia, anorexia nervosa, anxiety, generalized anxiety disorder, social anxiety disorder, body dismographic disorder, obsessive compulsive disorder, paranoid disorder, nightmares, agitation, post-traumatic stress disorder (PTSD), severe mood dysregulation, mental disorder such as depression or anxiety that leads to metabolic diseases such as obesity and depression associated with any of the above clinical conditions. Said schizophrenia is selected from the group consisting of paranoid schizophrenia, disorganized schizophrenia, undifferentiated schizophrenia, catatonic schizophrenia and residual schizophrenia.

Said schizophrenia, in the above method of treatment can be selected from adult schizophrenia and pediatric schizophrenia and may take the form of a negative symptom of schizophrenia, a positive symptom of schizophrenia and both.

In some embodiments, there is provided a method of treatment of a mental disorder in a patient in need thereof with a composition of the present disclosure, wherein the mental disorder is schizophrenia and the CB2 receptor selective agonist is beta caryophyllene (BCP) as sole active agent.

In some embodiments, there is provided a method of treatment of a mental disorder in a patient in need thereof with a composition of the present disclosure, wherein the mental disorder is schizophrenia, the CB2 receptor selective agonist is BCP and the at least one antipsychotic agent is selected from the group consisting of risperidone, paliperidone, paliperidone palmitate, aripiprazole, quetiapine, CBD and its analogs, THCV, brexpiprazole and combinations thereof.

In some embodiments, there is provided a method of treatment of a mental disorder in a patient in need thereof with the composition of the present disclosure, wherein the composition comprises a therapeutically effective amount of BCP as sole active agent in a self-emulsifying vehicle.

According to some embodiments, there is provided a method of treatment of a mental disorder in a patient in need thereof with a composition of the present disclosure, wherein said composition comprises a therapeutically effective amount of at least one CB2 receptor selective agonist in essentially pure form and optionally a therapeutically effective amount of at least one anti-psychotic agent in a self-emulsifying vehicle, wherein the composition is administered to a patient in need thereof from about once a month to about once every two months, to about once every three months, to about once every-four months, to about once every five months, to about once every six months, to about once per week, twice per week, 3 times per week, 4 times per week, 5 times per week, 6 times per week, once per day, twice per day or 3 times per day.

According to another embodiment, there is provided a method of treatment of a mental disorder in a patient in need thereof with a composition of the present disclosure, whereto said composition comprises a therapeutically effective amount of at least one CB2 receptor selective agonist in essentially pure form and optionally a therapeutically effective amount of at least one antipsychotic agent in a self-emulsifying vehicle and is administered twice per week to a patient in need thereof. Similarly, there is provided a method of treatment of a mental disorder in a patient in need thereof with the composition of the present disclosure, wherein said composition comprises a therapeutically effective amount of at least one CB2 receptor selective agonist in essentially pure form and optionally a therapeutically effective amount of at least one antipsychotic agent in a self-emulsifying vehicle, and is administered three times a week to a patient in need thereof.

According to another embodiment, there is provided a method of treatment of a mental disorder in a patient in need thereof with a composition of the present disclosure wherein the therapeutically effective amount of the composition comprising BCP as sole active and a self-emulsifying vehicle is administered to a patient in need thereof from about once a month to about once every two months, to about once every three months, to about once every four months, to about once every five months, to about once every six months, to about once per week, twice per week, 3 times per week, 4 times per week, 5 times per week, 6 times per week, once per day, twice per day, 3 times per day or 4 times per day.

According to an embodiment, there is provided a method of treatment of a mental disorder in a patient in need thereof with a composition of the present disclosure wherein the therapeutically effective amount of the composition comprising BCP as sole active and a self-emulsifying vehicle is administered twice per week or three times per week to a patient in need thereof.

In an embodiment, there is provided a method of treatment of a mental disorder in a patient in need thereof with a composition, in any mode of administration, including but not limited to administration in a slow-release/long-active formulations given on a daily basis, of the present disclosure wherein the average daily amount of said BCP or HU-308 or 4-0-methylhonokiol (MH) administered is in a range selected from the group consisting of 0.1-1 mg, 1-10 mg, 10-20 mg, 20-50 mg, 50-100 mg, 100-200 mg or 200-1000 mg, according to the patient's age and composition's effectiveness.

In an embodiment, there is provided a method of treatment of a mental disorder in a patient in need thereof with a delayed-release composition (such as compositions for a slow-release, slow-acting form of medication prepared as a capsule or a depot injection given for example but not limited by intramuscular injection, which are administrated every 1 week or once a month to up to every six months) of the present disclosure wherein the average amount of a single administration of said BCP administered is in a range selected from, the group consisting of 0.1-10 mg, 10-100 mg, 100-200 mg, 200-300 mg, 300-400 mg, 500-600 mg, 600-700 mg, 700-800 mg or 800-1000 mg, 1000-1500 mg, 1000-2000 mg, 2000-3000 mg, according to patient's age and composition's effectiveness. According to an embodiment, there is provided a method of treatment of a mental disorder in a patient in need thereof with a composition of the present disclosure, wherein said at least one antipsychotic agent is co-administered in a single dosage form together with said CB2 receptor modulator.

According to another embodiment, there is provided a method of treatment of a mental disorder in a patient in need thereof with a composition of the present disclosure, wherein said at least one antipsychotic agent is co-administered sequentially in a dosage form separate from said CB2 receptor selective agonist wherein in either order.

In some embodiments, there is provided the use of a therapeutically effective amount of at least one CB2 receptor modulator in substantially pure form in a self-emulsifying vehicle and optionally of a therapeutically effective amount of at least one antipsychotic agent in the manufacture of a composition for treating a mental disorder in a subject in need thereof.

In some embodiments, there is provided a method of treatment of a mental disorder in a patient in need thereof with a composition of this disclosure, wherein the at least one CB2 receptor selective agonist in substantially pure form is beta caryophyllene (BCP) as sole active agent and the mental disorder is bi-polar disorder, onset at any age.

In some embodiments, there is provided a method of treatment of a mental disorder in a patient in need thereof with a composition of the present disclosure wherein the at least one CB2 receptor selective agonist in substantially pure form is beta caryophyllene (BCP) as sole active agent and the mental disorder is depression, onset at any age.

According to some embodiments, there is provided a method of treatment of a mental disorder in a patient in need thereof with a composition of the present disclosure, wherein the at least one CB2 selective receptor agonist in substantially pure form is beta caryophyllene (BCP) as sole active agent and the mental disorder is anxiety, onset at any age.

In some embodiments, there is provided a stable self-emulsifying composition for treatment of mental disorders in a patient in need thereof, comprising a therapeutically effective amount of at least one CB2 receptor modulator, a self-emulsifying vehicle and optionally a therapeutically effective amount of at least one additional active agent selected from the group consisting of an antipsychotic agent and combinations thereof, wherein the active agents are substantially solubilized.

The above self-emulsifying composition upon dilution with water containing media or body fluid spontaneously forms an oil-in-water emulsion.

In some embodiments, there is provided the above self-emulsifying composition, wherein the at least one CB2 receptor modulator is selected from the group consisting of at least one CB2 receptor agonist or partial agonist, at least one CB2 receptor antagonist or inverse agonist, at least one CB2 receptor antagonist or inverse agonist winch is also a selective estrogen receptor modulator (SERM), at least one type of CB2 receptor allosteric modulator and combinations thereof.

In some embodiments, the at least one CB2 receptor agonist or partial agonist in the above composition is selected from the group consisting of BCP, HU-308, HU-433, HU-910, HU-914, CB 65, GP 1a, GP 2a, GW 405833, JWH 015, JWH 133, AM1241, L759,656L-759,633, MDA 19, SER 601, BML-190, N-alkylamide, rutamarin, diindolylmethane (DIM), cannabinor (PRS-211,375), 2-arachidonoylglycerol, anandamide, CP55940, delta-9-THC, W1N55212-2, HU-210, cannabigerol (CBG), 11-hydroxy-Δ9-tetrahydrocannabinol (11-OH-THC), delta-8-THC, 11-OH-delta-8-THCV, ajulemic acid, delta-8-THC-11-oic acid, cannabinol (CBN), cannabilactones, AM1714, AM1710; analogs thereof, derivatives thereof, metabolites thereof and combinations thereof.

In some embodiments, the at least one CB2 receptor antagonist or inverse agonist is selected from the group consisting of AM630, JTE-907, SR144528, COR170, 4-0-methylhonokiol GS12021 (4-0methylhonokiol analogue), cannabinol, 01238, 01184, cannabidiol (CBD) analogs thereof, derivatives thereof and combinations thereof.

In some embodiments, the at least one CB2 receptor allosteric modulator is selected from the group consisting of dihydrogambogic acid, garcinolic acid, (-)-5′-dimethylheptyl-cannabidiol (DMH-CBD) and analogs thereof, derivatives thereof and combinations thereof.

In some embodiments, the at least one CB2 receptor modulator which is also a selective estrogen receptor modulator (SERM) is selected from the group consisting of raloxifene, bazedoxifen, lasofoxifene, tamoxifen, afimoxifene, arzoxifene, ormeloxifene, toremifene, ospemifene, analogs thereof, derivatives thereof and combinations thereof.

In some embodiments, the at least one antipsychotic agent is selected from the group consisting of one or more of a butyrophenone type antipsychotic agent selected from the group consisting of haloperidol, droperidol, benperidol, trifluperidol, melperone, lenperone, azaperone, domperidone, butyrophenone, fluanisone, penfluridol, pipamperone, spiperone, nonaperone, bromperidol and timiperone, a diphenylbutylpiperidine type antipsychotic agent selected from the group consisting of luspirilene, penfluridol, pimozide, clopimozide, fluspirilene, penfluridol, a phenothiazine type antipsychotic acid agent selected from the group consisting of acepromazine, chlorpromazine, cyamemazine, dixyrazine, fluphenazine, levomepromazine, mesoridazine, perazine, pericyazine, perphenazine, pipotiazine, prochlorperazine, promazine, promethazine, prothipendyl, thioproperazine, thioridazine and trifluoperazine and triflupromazine, a thioxanthene type antipsychotic agent selected from the group consisting of chlorprothixene, clopenthixol, flupentixol, thiothixene and zuclopenthixol and/or an atypical antipsychotic agent including, but not limited to one or more of an atypical antipsychotic agent usually belonging to the D2 antagonist/inverse agonist, 5-HT2A antagonist/inverse agonist types selected from the group consisting of amisulpride, amoxapine, asenapine, cariprazine, clozapine, blonanserin, iloperidone, lurasidone, melperone, nemonapride, olanzapine, paliperidone, paliperidone palmitate, perospirone, quetiapine, remoxipride, risperidone, sertindole, sultopride, trimipramine, ziprasidone, ITI-007, pimavanserin (ACP-103; 5-HT2A antagonist), and combinations thereof, and/or an atypical, antipsychotic agent including, but not limited to one or more of an atypical antipsychotic agent usually belonging to the D2 partial agonist types selected from the group consisting aripiprazole and its metabolites OPC-14857, DM-1458, DM-1451, DM-1452, DM-1454 and DCPP, brexpiprazole and RP5063 (RP5000) and combinations thereof and/or a cannabinoid exhibiting antipsychotic activity selected from the group consisting of tetrahydrocannabivarin (THCV—CB1 antagonist, CB2 receptor partial agonist), cannabidiol (CBD—CB1/CB2/(GPR55/ABn-CBD antagonist/inhibitor) and cannabigerol (CBG—CB1/CB2 partial agonist), and their analogs and derivatives and combinations thereof.

In some embodiments, the stable self-emulsifying drug delivery composition of this invention comprises at least one oil, at least one surfactant HLB<9, at least one surfactant HLB>13, at least one co-surfactant, at least one antioxidant and/or free-radical scavenger, at least one CB2 receptor modulator and optionally an antipsychotic agent, and combinations thereof.

In some embodiments, the stable self-emulsifying drug delivery composition of this invention comprises:

from about 10% w/w to about 50% w/w of an oil selected from the group consisting of medium chain triglycerides, propylene glycol dicaprilate/dicaprate, medium, chain mono- and diglycerides, acetylated mono- and diglycerides, sesame oil and olive oil and combinations thereof,

from about 20% w/w to about 50% w/w of a surfactant HLB<9 selected from the group consisting of oleoyl polyoxyl-6 glycerides, linoleyl polyoxyl-6 glycerides (20-40%), Polysorbate 85 (Tween-85) polyoxyethylene (20-40%), sorbitan trioleate (5-15%), Span-80 (sorbitan monooleate) (5-25%), polyglyceryl-3 dioleate (15-35%) and glycerin, monolinoleate (10-35%), Polysorbate 80 (Tween-80) polyoxyethylene (20-40%), Polysorbate 60 (Tween-60) polyoxyethylene (20-40%),

from about 5% w/w to about 50% w/w of a surfactant HLB>13 selected from the group consisting of polyoxylated castor oil (5-40%), PEG 40 hydrogenated castor oil, PEG-15 hydroxystearate (5-25%) and caprylocaproyl polyoxyl-8 glycerides (10-20%).

from about 5% w/w to about 25% w/w of a surfactant HLB>13 selected from the group consisting of PEG-20 sorbitan monostearate, PEG-20 sorbitan monooleate (5-25%) and PEG 40 stearate (5-25%),

from about 0.5% w/w to about 15% w/w of a co-surfactant selected from the group consisting of any lecithin (2-15% w/w), soy lecithin (>=75% phosphatidylcholine in oil, 1-10% w/w), soy lecithin PC content >50% (2-15% w/w), egg lecithin E-60 or E-80 (1-5% w/w) and distearoylphosphatidylcholine (0.5-3% w/w),

from about 0.1% w/w to about 5% w/w of an antioxidant or free radical scavenger selected from the group consisting of d-alpha-tocopherol (1-10% w/w), dl-alphatocopherol (2-15% w/w), dl-alpha-tocopheryl acetate (2-15% w/w), mixed tocopherols (alpha, beta, gama—1-10% w/w), d-alpha-tocopheryl acetate (2-15% w/w), butylated hydroxyanisole (BHA, 0.01-0.5% w/w), tocophersolan (TPGS, tocopherol PEG ester succinate) (2-10% w/w) and combinations thereof,

from about 5% w/w to about 10% w/w of ethyl alcohol,

from about 1% w/w to about 20% w/w of at least one CB2 receptor modulator in substantially pure form and optionally from about 0.1% w/w to about 5% w/w of at least one antipsychotic agent.

In an embodiment, there are provided stable self-emulsifying drug delivery compositions, comprising:

from about 30% w/w to about 50% w/w capric/caprylic triglycerides

from about 30% w/w to about 50% w/w oleoyl polyoxyl-6 glycerides

from about 5% w/w to about 35% w/w polyoxylated castor oil

from about 7% w/w to about 15% w/w PEG-20 sorbitan monostearate

from about 2% w/w to about 10% w/w soy lecithin (75% phosphatidylcholine in oil)

from about 1% w/w to about 15% w/w d-alpha tocopherol and/or tocopherol acetate

from about 1% w/w to about 20% w/w of at least one CB2 receptor modulator and

optionally

from about 0.1% w/w to about 5% w/w of at least one antipsychotic agent.

In another embodiment, there is provided a stable self-emulsifying drug delivery composition of this invention, wherein the at least one CB2 receptor agonist is beta-caryophyllene (BCP) as sole active agent in a self-emulsifying vehicle.

In yet another embodiment, there is provided a stable self-emulsifying drug delivery composition of this invention, wherein the at least one CB2 receptor agonist is beta-caryophyllene (BCP) and the at least one antipsychotic agent is selected from the group consisting of risperidone, paliperidone, paliperidone palmitate, aripiprazole, quetiapine, CBD, THCV, CBG, brexpiprazole their derivatives and analogs and combinations thereof.

According to some embodiments, there is provided a composition of this invention, wherein the at least one CB2 receptor agonist is beta-caryophyllene (BCP) and the at least one antipsychotic agent is selected from the group consisting of 10-98% CBD, 10-98% THCV, 10-98% CBG and combinations thereof.

In another embodiment, there is provided a stable self-emulsifying drug delivery composition of this invention, wherein the at least one CB2 receptor agonist is beta-caryophyllene (BCP) as sole active agent in a self-emulsifying vehicle and the mental disorder is schizophrenia of all types, onset at any age.

According to an embodiment, there is provided a composition of this invention, wherein the at least one CB2 receptor agonist is BCP and the at least one additional active agent is selected from the group consisting of alpha-humulene, copaene, eugenol, δ-cadinene, BCP oxide and combinations thereof.

According to another embodiment, there is provided a composition of this invention, in which said BCP comprises from 1% w/w to 15% w/w alpha-humulene and from 0.1%-2% w/w each of copaene, eugenol, δ-cadinene, BCP oxide, derivatives thereof, analogs thereof and combinations thereof.

In some embodiments, there is provided a composition of this invention, wherein the at least one CB2 receptor selective agonist in substantially pure form is beta caryophyllene (BCP), the at least one antipsychotic agent is selected from the group consisting of risperidone, paliperidone, paliperidone palmitate, aripiprazole, quetiapine, CBD, THCV CBG, brexpiprazole; derivatives thereof, analogs thereof and combinations thereof and the mental disorder is schizophrenia.

In some other embodiments, there is provided a composition of any of claims 12-18, wherein said BCP comprises either one of the two BCP isomers E-BCP and Z-BCP in substantially pure form or mixtures thereof and wherein substantially free of BCP oxide and a-humulene.

According to an embodiment, there is provided a composition of this invention, wherein said BCP comprises substantially the isomer E-BCP and is optionally free of BCP oxide and a-humulene.

According to another embodiment, there is provided a composition of this invention, wherein said BCP comprises the substantially pure isomer Z-BCP and is optionally free of BCP oxide and a-humulene.

In some embodiments there is provided a composition of this invention, wherein the at least one CB2 receptor agonist is [(1R,2R,5R)-2-[2,6-dimethoxy-4-(2-methyloctan-2-yl) phenyl]-7,7-dimethyl-4-bicyclo[3.1.1]hept-3-enyl] methanol (HU-308) as sole active agent.

In some other embodiments there is provided a composition of this invention, wherein the at least one CB2 receptor agonist is HU-308 and the at least one antipsychotic agent is selected from the group consisting of risperidone, paliperidone, paliperidone palmitate, aripiprazole, quetiapine, CBD, THCV, CBG, brexpiprazole; derivatives thereof, analogs thereof and combinations thereof.

According to some embodiments, there is provided a composition of this disclosure, wherein the at least one CB2 receptor inverse agonist is 4-0-methylhonokiol (MH), and the at least one additional active agent is selected from the group consisting of eugenol, caryophyllene oxide and combinations thereof.

In some embodiments, there is provided a composition of this invention, wherein the at least one CB2 receptor selective agonist is 4-0-methylhonokiol as sole active agent and the mental disorders are tic disorders, repetitive behavior disorders of all types, onset at any age.

In some embodiments, the stable composition of this disclosure is stabilized by addition of an antioxidant, a free-radical scavenger or a combination thereof.

In an embodiment, there is provided a composition of the instant disclosure, wherein formulated for oral, parenteral, topical, intranasal, vaginal or rectal administration.

The above oral composition can be formulated as a spray, inhalation, capsule, suspension, solution, emulsion or syrup.

The above topical composition can be formulated as a transdermal gel, cream, patch or topical spray.

The above intranasal composition can be formulated as a nasal spray.

In some embodiments, there is provided a method of treatment of a mental disorder in a patient in need thereof, by administration of a composition of this disclosure, comprising a therapeutically effective amount of at least one CB2 receptor modulator, a self-emulsifying vehicle and optionally a therapeutically effective amount of at least one antipsychotic agent and combinations thereof.

In another embodiment, there is provided a method of treatment of a mental disorder in a patient in need thereof, by administration of a composition of this invention, comprising a therapeutically effective amount of BCP and from 1% w/w to 15% w/w alpha-humulene and from 0.1% w/w-2% w/w each of copaene, eugenol, δ-cadinene, BCP oxide, caryophyllene oxide and their derivatives and analogs and combinations thereof, a self-emulsifying vehicle and optionally a therapeutically effective amount of at least one antipsychotic agent and combination thereof.

According to an embodiment, there is provided a method of treatment of a mental disorder in a patient in need thereof, wherein the mental disorder is schizophrenia, by administration of a composition comprising a therapeutically effective amount of BCP and from 1% to 15% alpha-humulene and from 0.1-2% each of copaene, eugenol, δ-cadinene, BCP oxide, caryophyllene oxide and their derivatives and analogs and combinations thereof, a self-emulsifying vehicle and optionally a therapeutically effective amount of either at least one antipsychotic agent and combination thereof, the CB2 receptor selective agonist is beta caryophyllene (BCP) and optionally at least one additional active agent selected from the group consisting of alpha-humulene, copaene, eugenol, δ-cadinene, BCP oxide and combinations thereof.

According to another embodiment, there is provided a method of treatment of a mental disorder in a patient in need thereof, wherein the mental disorder is bi-polar disorder, onset at any age, by administration of a composition comprising at least one CB2 receptor selective agonist, wherein the at least one CB2 receptor selective agonist is beta caryophyllene (BCP) and optionally at least one additional active agent selected from the group consisting of alpha-humulene, copaene, eugenol, δ-cadinene, BCP oxide and combinations thereof.

According to another embodiment, there is provided a method of treatment of a mental disorder in a patient in need thereof, wherein the mental disorder is depression, onset at any age by administration of a composition comprising at least one CB2 receptor selective agonist which is beta caryophyllene (BCP) and optionally at least one additional active agent alpha-humulene, copaene, eugenol, δ-cadinene, BCP oxide and combinations thereof.

According to another embodiment, there is provided a method of treatment of a mental disorder in a patient in need thereof, wherein the mental disorder is anxiety, onset at any age, by administration of a composition comprising at least one CB2 receptor selective agonist which is beta caryophyllene (BCP) as sole active agent and optionally at least one agent is selected from the group consisting of alpha-humulene, copaene, eugenol, δ-cadinene, BCP oxide and combinations thereof.

In an embodiment, there is provided a method of treatment of a mental disorder in a patient in need thereof, by administration of a composition comprising a therapeutically effective amount of BCP and at least one antipsychotic agent selected from the group consisting of risperidone, paliperidone, paliperidone palmitate, aripiprazole, quetiapine, CBD and its derivatives and analogs, THCV, CBGV, brexpiprazole and combinations thereof.

In another embodiment, there is provided a method of treatment of a mental disorder in a patient in need thereof, by administration of a composition of this invention, comprising at least one CB2 receptor modulator selected from the group consisting of at least one CB2 receptor agonist or partial-agonist, at least one CB2 receptor antagonist of inverse agonist, at least one CB2 receptor antagonist or inverse agonist which is also a selective estrogen receptor modulator (SERM), at least one type of CB2 receptor allosteric modulator and combinations thereof.

In another embodiment, there is provided a method of treatment of a mental disorder in a patient in need thereof, by administration of a composition of this invention, comprising a CB2 receptor selective agonist or partial agonist selected from: the group comprising BCP, HU-308, HU-433, HU910, HU-914, CB 65, GP 1a, GP 2a, GW 405833, JWH 015, JWH 133, AM1241, L759,656, L759,633, MDA 19, SEE 601, BML-190, N-alkylamide, rutamarin, diindolylmethane (DIM) and analogs, CBG, 11-hydroxy-Δ9-tetrahydrocannabinol (11-OH-THC), delta-8-THC, 11-OH-delta-8-THCV, ajulemic acid, delta-8-THC-11-oic acid, cannabinol, cannabilactones, AM1714, AM1710; and analogs, derivatives and combinations thereof.

In yet another embodiment, there is provided a method of treatment of a mental disorder in a patient in need thereof, by administration of a composition of this invention, comprising a CB2 receptor antagonist or inverse agonist selected from the group consisting of AM630, JTE-907, SR144528, COR170, 4-0-methylhonokiol, GS12021 (4-0-methylhonokiol analog), cannabinol, 01238, 01184, cannabidiol (CBD); and analogs, derivatives or combinations thereof.

In some embodiments, there is provided a method of treatment of this disclosure, wherein the at least one antipsychotic agent is selected from the group consisting of one or more of a butyrophenone type antipsychotic agent selected from the group consisting of haloperidol, droperidol, benperidol, trifluperidol, melperone, lenperone, azaperone, domperidone, butyrophenone, fluanisone, penfluridol, pipamperone, spiperone, nonaperone, bromperidol and timiperone, a diphenylbutylpiperidine type antipsychotic agent selected from the group consisting of luspirilene, penfluridol, pimozide, clopimozide, fluspirilene, penfluridol, a phenothiazine type antipsychotic acid agent selected from the group consisting of acepromazine, chlorpromazine, cyamemazine, dixyrazine, fluphenazine, levomepromazine, mesondazine, perazine, pericyazine, perphenazine, pipotiazine, prochlorperazine, promazine, promethazine, prothipendyl, thioproperazine, thioridazine and trifluoperazine and triflupromazine, a thioxanthene type antipsychotic agent selected from the group consisting of chlorprothixene, clopenthixol, flupentixol, thiothixene and zuclopenthixol and/or an atypical antipsychotic agent including, but not limited to one or more of an atypical antipsychotic agent usually belonging to the D2 antagonist/inverse agonist, 5-HT2A antagonist/inverse agonist types selected from the group consisting of amisulpride, amoxapine, asenapine, cariprazine, clozapine, blonanserin, iloperidone, lurasidone, melperone, nemonapride, olanzapine, paliperidone, paliperidone palmitate, perospirone, quetiapine, remoxipride, risperidone, sertindole, sultopride, trimipramine, ziprasidone, ITI-007, pimavanserin (ACP-103; 5-HT2A antagonist), and combinations thereof, and/or an atypical antipsychotic agent including, but not limited to one or more of an atypical antipsychotic agent usually belonging to the D2 partial agonist types selected from the group consisting aripiprazole and its metabolites OPC-14857, DM-1458, DM-1451, DM-1452, DM-1454 and DCPP, brexpiprazole and RP5063 (RP5000) and combinations thereof and/or a cannabinoid exhibiting antipsychotic activity selected from the group consisting of tetrahydrocannabivarin (THCV—CB1 antagonist, CB2 receptor partial agonist), cannabidiol (CBD—CB1/CB2/GPR55/ABN-CBD antagonist/inhibitor) and cannabigerol (CBG—CB1/CB2 partial agonist); analogs thereof, derivatives thereof and combinations thereof.

In another embodiment, there is provided a method of treatment of a mental disorder in a patient in need thereof, by administration of a composition of this invention, wherein the disease or mental disorder is selected from the group consisting of schizophrenia, schizoaffective disorder, bipolar disorder I and II, unipolar disorder, multiple personality disorder, psychotic disorders, depression, psychotic depression, depressive disorders, major depressive disorder, stereotypic movement disorder, autism spectrum disorders, obsessive-compulsive disorder (OCD), bacterial-induced tic disorder, pediatric autoimmune neuropsychiatric disorders associated with streptococcal infections (PANDAS), chorea (Sydenham's chorea (SC), chorea minor, chorea gravidarum, drug-induced chorea), drug-induced repetitive behaviors, akathisia, dyskinesias, Wernicke-Korsakoff syndrome, Tourette's syndrome, tic disorders, epilepsy, anxiety disorders, autistic spectrum disorder, enuresis, addiction, withdrawal symptoms associated with addiction, Asperger syndrome, oppositional defiant disorder, behavioral disturbance, agitation, psychosis/agitation associated with Alzheimer's disease, psychosis associated with Parkinson's disease, psychosis associated with drug of abuse, psychosis associated with psychedelic drug abuse, LSD-induced psychosis, steroid-induced schizophrenia, steroid-induced psychosis, Capgras syndrome; Fregoli syndrome; Cotard, personality disorders, borderline personality disorder, avoidant personality disorder, attention-deficit/hyperactive disorder (ADHD, ADD, HD), mania, dementia, anorexia, anorexia nervosa, anxiety, generalized anxiety disorder, social anxiety disorder, body dismographic disorder, obsessive compulsive disorder, paranoid disorder, nightmares, agitation, post-traumatic stress disorder (PTSD), severe mood dysregulation, developmental coordination disorder, stereotypic movement disorder, bacterial-induced tic disorder, pediatric autoimmune neuropsychiatric disorders associated with streptococcal infections (PANDAS), chorea (Sydenham's chorea (SC), chorea minor, chorea gravidarum, drug-induced chorea), drug-induced repetitive behaviors, akathisia, dyskinesias, Wernicke-Korsakoff syndrome, neuroinflammatory diseases, neurodegenerative diseases, liver associated-diseases, hepetatis, alcohol-related liver disease, fibromyalgia, gastrointestinal diseases, inflammatory bowel disease, Crohn's disease, ulcerative colitis, cancer, mental disorder such as depression or anxiety that leads to metabolic diseases such as obesity and depression associated with any of the above clinical conditions and cognitive deficits associated with any of the above clinical conditions and combinations thereof, wherein the disease is an acute, transient or chronic disease.

In another embodiment, there is provided a method of treatment of a mental disorder in a patient in need thereof, by administration of a composition of this invention, wherein said mental disorder is schizophrenia and wherein said schizophrenia includes any symptom and its onset is at any age.

In some embodiments, there is provided a method of treatment of a mental disorder by administration of a composition of this invention, wherein said composition comprises, a therapeutically effective amount of at least one CB2 receptor modulator and optionally a therapeutically effective amount of at least one antipsychotic agent and combination thereof, in a self-emulsifying vehicle and is administered to a patient in need thereof from about once a month to about once every two months, to about once every three months, to about once every four months, to about once every five months, to about once every six months, to about once per week, twice per week, 3 times per week, 4 times per week, 5 times per week, 6 times per week, once per day, twice per day, 3 times per day or 4 times per day.

In some other embodiments, there is provided a method of treatment of a mental disorder by administration of a composition of this invention, the composition comprising a therapeutically effective amount of at least one CB2 receptor modulator and optionally a therapeutically effective amount of at least one antipsychotic agent, at least one GPR55 modulator, at least one anti-inflammatory agent and combinations thereof, in a self-emulsifying vehicle, wherein the composition is administered twice per week to a patient in need thereof.

In an embodiment, there is provided a method of treatment of a mental disorder by administration of a composition of this invention, the composition comprising a therapeutically effective amount of at least one CB2 receptor selective modulator and optionally an antioxidant, a therapeutically effective amount of at least one antipsychotic agent and combinations thereof, in a self-emulsifying vehicle, wherein the composition is administered once per week, twice per week, three times per week to a patient in need thereof.

In some embodiments, there is provided a method of treatment of a mental disorder by administration of a composition of this invention, wherein the composition comprises a therapeutically effective amount BCP, HU-308 or MH as sole active and a self-emulsifying vehicle, and wherein the composition is administered to a patient in need thereof from about once a month to about once every two months, to about once every three months, to about once every four months, to about once every five months, to about once every six months, to about once per week, twice per week, 3 times per week, 4 times per week, 5 times per week, 6 times per week, once per day, twice per day, 3 times per day or 4 times a day.

In some embodiments, there is provided a method of treatment of a mental disorder by administration of a composition of this invention, wherein the average daily amount of said either BCP, HU-308, 4-0-methylhonokiol (MH) administered in any daily mode of administration, including but not limited to administration in delayed-release formulations given on a daily basis, is in a range selected from the group consisting of 0.01-0.1 mg, 0.1-1 mg 1-10 mg, 10-25 mg, 25-100 mg, 100-1000 mg, according to the age and the effectiveness of the composition.

In some embodiments, there is provided a method of treatment of a mental disorder by administration of a composition of this invention, wherein the average amount of a single administration of a delayed-release delivery composition is selected from compositions for slow-release, delayed release drugs formulated as a capsule or as a depot injection given either orally or mostly by injection, administrated once a week or once a month to up to every six months comprising BCP, HU-308, or 4-0-methylhonokiol (MH), administered in amount selected from 0.1-10 mg, 10-25 mg, 25-100 mg, 100-1000 mg or 100-3000 mg, according to patient's age and composition's effectiveness.

In some other embodiments, there is provided a method of treatment of a mental disorder by administration of a composition of this invention, wherein said at least one antipsychotic agent and combinations thereof, is co-administered in a single dosage form together with said CB2 receptor modulator.

According to an embodiment, there is provided a method of treatment of a mental disorder by administration of a composition of this invention to a patient in need thereof, wherein the at least one antipsychotic agent, is co-administered sequentially in a dosage form separate from said CB2 receptor selective agonist in either order.

In some embodiments, there is provided a use of a therapeutically effective amount of at least one CB2 receptor modulator in substantially pure form in a self-emulsifying vehicle and optionally of a therapeutically effective amount of at least one antipsychotic agent, in the manufacture of a composition for treating a mental disorder in a subject in need thereof.

In some other embodiments, there is provided a composition for the treatment of a mental disorder in a patient in need thereof, wherein formulated as a stable self-emulsifying drug delivery system comprising:

from about 0.01% w/w to about 0.2% w/w butylated hydroxytoluene,

from about 1% w/w to about 40% w/w Tween-60 (Polysorbate 60 NF),

from about 1% w/w to about 40% w/w Tween-80 (Polysorbate 80 NF),

from about 1% w/w to about 15% w/w Span 80 (Sorbitan monooleate) NF,

from about 1% w/w to about 15% w/w Tocophersolan (TPGS, Tocopherol PEG ester succinate).

from about 1% w/w to about 30% w/w Labrafil M1944 CS,

from about 1% w/w to about 15% w/w Lecithin (Phospholipon 80),

from about 1% w/w to about 15% w/w Ethyl alcohol anhydrous, and optionally from about 0.1% w/w to about 5% w/w of at least one antipsychotic agent.

Exemplary embodiments of the teachings herein are discussed herein below with reference to specific materials, methods and examples. The material, methods and examples discussed herein are illustrative and not intended to be limiting. In some embodiments, methods and materials similar or equivalent to those described herein are used in the practice or testing of embodiments of the invention. It is to be understood that the invention is not necessarily limited in its application to the details of construction and the arrangement of the components and/or methods set forth in the following description and/or illustrated in the drawings. The invention is capable of other embodiments or of being practiced or carried out in various ways.

### EXAMPLES

### Materials and Methods

BCP was obtained from Sigma-Aldrich (St Louis, Mo., USA), catalogue Nr. W225207 (assay not indicated) and further purified using preparative HPLC (HP1090 series; column, PEGASIL ODS (Senshu Sci. i.d. 10×250 mm); solvent, 70% CH3OH; How rate, 2.0 mL/min; detection, UV 220 nm] to remove other sesquiterpenes.

Purified BCP batches were analyzed by GC-MS analysis;

Batch 1: Total BCP—98%; 95% E-BCP, 3% Z-BCP, 1% BCP oxide and traces of a-humulene.

Batch 2: Total BCP—about 85%, about 13% alpha-humulene, about 1% copaene, about 0.3% eugenol, about 0.3% δ-cadinene and about 0.3% BCP oxide

Phencyclidine (PCP), Cremophor EL and DMSO were obtained from Sigma-Aldrich (St. Louis, Mo., USA).

### Animal Model of Schizophrenia:

The mouse model of schizophrenia was established. Phencyclidine (PCP), an NMDA antagonist which induces schizophrenia and psychotic effects in humans, was administered to murine pups (injection of 5 mg/kg in saline) on postnatal days 3, 5, 7, 9, 11, 13, and 16 (or 3 times a week, on alternated days, for 2 weeks). This treatment induces long-lasting schizophrenic-like effects in mice that lasted into adulthood. The therapeutic effects of betacaryophyllene, a dietary cannabinoid and CB2 receptor selective agonist, in accordance with the teachings herein were evaluated.

### Example 1

Oral 16% BCP composition in a SEDDS (self-emulsifying drug delivery system) vehicle.

### Preparation of the SEDDS Vehicle

### Vehicle

| Component | gram | % |
|---|---|---|
| MCT oil (Capric/caprylic triglycerides) NF | 38.4 | 38.40% |
| Labrafil M1944CS EP (Oleoyl polyoxyl-6 glycerides) | 38.0 | 38.00% |
| Kollliphor EL NF (PEG 40 castor oil) | 7.25 | 7.25% |
| Polysorbate 60 (Tween-60) | 11.8 | 11.80% |
| Soy lecithin (Phosal 75 SA) | 2.95 | 2.95% |
| dl-alpha-Tocopherol USP | 1.6 | 1.60% |
| Total: | 100 | 100.0% |

The ingredients dl-alpha tocopherol and Phosal 75SA were stored in a refrigerator. dl-Alpha tocopherol and Phosal 75SA were removed from refrigerator and allowed to reach room temperature while tightly closed.

Labrafil M1944CS and Polysorbate 60 were heated to 50-55° C. until each product becomes a clear and homogenous liquid.

The following ingredients were weighed into a 200 ml glass beaker weigh in the following order: dl-alpha Tocopherol (1.760 g), Phosal 75SA (3.245 g), Kolliphor EL (7.975 g), Polysorbate 60 (12.980 g), Labrafil M1944CS (41.800 g) and Capric/caprylic triglycerides (42.245 g)—Total: 110.00 g (A—0.962 g/ml).

The beaker was covered and heated to 45-50° C. until all ingredients are completely melted. The obtained liquid was mixed using a magnetic stirrer at medium/low speed until a homogenous liquid SEDDS vehicle was formed (10-20 minutes).

The SEDDS vehicle obtained as a hazy liquid was transferred to amber glass storage bottles and the head space was flushed with nitrogen. The bottles were tightly closed, sealed and stored in a refrigerator at +2-8° C.

### Preparation of the BCP Oral Composition in a SEDDS Vehicle

Composition (16% BCP)

| Component | gram | % |
|---|---|---|
| MCT oil | 32.26 | 32.26% |
| Labrafil M1944CS | 31.92 | 31.92% |
| Kolliphor EL | 6.09 | 6.09% |
| Tween 60 | 9.91 | 9.91% |
| Phosal 75SA | 2.48 | 2.48% |
| BCP (batch 1 or 2) | 16.00 | 16.00% |
| dl-alpha-Tocopherol | 1.34 | 1.34% |
| Total: | 10.000 | 100.00% |

The SEDDS vehicle was stored in a refrigerator. The active agent BCP was stored in a freezer.

The vehicle and the active were removed from storage, allowed to reach room temperature while tightly closed, then warmed to 35-40° C. using a water bath. The vehicle was shaken to homogenize it.

SEDDS vehicle (84.0 g) was weighed into an Erlenmeyer flask with a stopper and BCP (16.0 g) was added to it. The flask was closed and mixed using a magnetic stirrer for 10-15 minutes at low speed until a homogenous mixture was formed.

The oral composition obtained was slightly cloudy/opalescent.

The above oral composition is filled into capsules or diluted with water, as per need.

### Particle Size Analysis

Particle size was measured at 25° C. using dynamic light scattering analyzer Zetasizer Nano ZS (Malvern. Instruments Ltd., UK) after dilution of the sample of Example 1 (16% BCP) with saline 1:1000. Results for intensity are presented on Chart 1.

CHART 1
Particle size analysis - Example 1 (16% BCP)
Results

| Diam. (nm) | | % Intensity | Width (nm) |
|---|---|---|---|
| Z-Average (d, nm): 212 | Peak 1:257 | 98.0 | 130 |
| PdI: 0.260 | Peak 2:5020 | 2.0 | 597 |

The water-diluted composition was found to be a submicron emulsion with average particle size of 260 nm and wide size distribution (50-800 nm).

The compositions in Examples 2-11 below were prepared in a, way similar to Example 1, using the quantities indicated in the Tables.

### Prophetic Examples 2-4

Vehicle and compositions tube A

| | VEHICLE A | | A1 Example 2 | | A2 Example 3 | | A3 Example 4 | |
|---|---|---|---|---|---|---|---|---|
| | mg | % | mg | % | mg | % | mg | % |
| Cremophor EL | 2320 | 11.9 | 2320 | 10.8 | 2320 | 9.82 | 2320 | 9.23 |
| Labrasol | 2150 | 11.0 | 2150 | 10.1 | 2150 | 9.0 | 2150 | 8.55 |
| Phosal MCT 53 | 1200 | 6.2 | 1200 | 5.61 | 1200 | 5.0 | 1200 | 4.77 |
| Acetylated mono/ | 13790 | 70.9 | 13790 | 64.4 | 13790 | 58.0 | 13790 | 54.83 |
| diglycerides | | | | | | | | |
| BCP | 0 | | 1945 | 9.09 | 4325 | 18.18 | 5340 | 21.23 |
| Alcohol | | | | | | | 350 | 1.39 |
| Total: | 19460 | 100 | 21405 | 100 | 23785 | 100 | 25150 | 100 |

| | | | | |
|---|---|---|---|---|
| Dilution with | + + + | + + + | + + + | + + |
| water media | | | | |

### Prophetic Examples 5-6

Vehicle and compositions type B

| | VEHICLE B | | B1 Example 5 | | B2 Example 6 | |
|---|---|---|---|---|---|---|
| | mg | % | mg | % | mg | % |
| Olive oil | 2000 | 64.7 | 2000 | 54.4 | 2000 | 38.62 |
| Tween-60 | 570 | 18.4 | 570 | 15.5 | | |
| Tween-85 | | | | | 300 | 5.79 |
| Span-80 | | | | | 660 | 12.75 |
| Cremophor EL | 290 | 9.4 | 290 | 7.9 | 600 | 11.59 |
| Phosal MCT 53 | 160 | 5.2 | 160 | 4.4 | 550 | 9.66 |
| Tocopherol (mix) | 70 | 2.3 | 70 | 1.9 | | |
| d-Tocopherol | | | | | 160 | 3.09 |
| BCP | 0 | 0.0 | 587 | 16.0 | 958 | 18.5 |
| | 3090 | 100 | 3677 | 100 | 5178 | 100 |

| | | | |
|---|---|---|---|
| Dilution with | + + ± | + + + | + + |
| water media | | | |

### Prophetic Examples 7-11

| | Example 7 | | Example 8 | | Example 8 | | Example 10 | | Example 11 | |
|---|---|---|---|---|---|---|---|---|---|---|
| Component | gram | % | gram | % | gram | % | gram | % | gram | % |
| Labrafac PG | 18 | 26.9 | | | | | 15.4 | 20.7 | 20 | 26.2 |
| (Propylene glycol | | | | | | | | | | |
| Labrafil M1925CS EP | 20 | 29.9 | 12 | 16.2 | 18 | 26.1 | | | | |
| (Linoleyl polyoxyl-6 | | | | | | | | | | |
| glycerides) | | | | | | | | | | |
| Plurol Oleique CC497 | | | 22 | 29.6 | | | 24 | 32.3 | 10 | 13.1 |
| (Polyglyceryl3 dioleate) | | | | | | | | | | |
| Maisine 35-1 | | | | | 16 | 23.2 | | | 10 | 13.1 |
| Kolliphor EL | | | 14 | 18.9 | 11 | 15.9 | 10.2 | 13.7 | | |
| Polysorbate 80 | | | 10.2 | 13.7 | 9 | 13.0 | 11.5 | 15.5 | 12.3 | 16.1 |
| Solutol HS-15 | 6 | 9.0 | | | | | | | 9.8 | 12.8 |
| PEG 40 stearate | 12 | 18.0 | | | | | | | | |
| Egg lecithin E-60 | 2 | 3.0 | 2.23 | 3.0 | | 0.0 | 1.6 | 2.2 | 1.89 | 2.5 |
| Distearoyl | | | | | 0.8 | 1.2 | | | | |
| phosphatidylcholine | | | | | | | | | | |
| BUT | | | | | 0.25 | 0.4 | | | | |
| dl-alpha-Tocopherol | 0.8 | 1.2 | 1.6 | 2.2 | 1.6 | 2.3 | 0.9 | 1.2 | 0.98 | 1.3 |
| BCP | 8 | 12.0 | 12.2 | 16.4 | 12.4 | 18.0 | 10.8 | 14.5 | 11.5 | 15.0 |
| Total: | 66.8 | 100 | 74.23 | 100.0 | 69.05 | 100 | 74.4 | 100 | 76.47 | 100.0 |

| | | | | | |
|---|---|---|---|---|---|
| Dilution with | + + | − + | + + | + + | + |
| water media | | | | | |

### Prophetic Example 12

### Liquid Composition for Oral Administration

For 1 teaspoon (5.0 g, approx. 5 ml) contains

| | |
|---|---|
| MCT oil | 1500 mg |
| Labrafil M1944CS | 1550 mg |
| Solutol HS-15 | 300 mg |
| Polysorbate 60 | 500 mg |
| Lecithin (75% PC) | 135 mg |
| Beta-caryophyllene (#1 #2) | 800 mg |
| dl-alpha-Tocopherol | 60 mg |
| Ethyl alcohol | 150 mg |
| Sucralose | 5 mg |
| | 5000 mg |

### Preparation:

Melt Polysorbate 60 and Solutol HS-15 at 45° C. and combine surfactants in an appropriate vessel.

Add MCT oil, Labrafil, Lecithin and Tocopherol, mix slowly until homogenous mixture is obtained. Cool the mixture to room temperature.

Add beta-caryophyllene and mix slowly for 10 minutes.

Separately dissolve Sucralose in ethyl alcohol (USP grade) at 45° C. Add solution to the mixture and mix slowly for 10 minutes. Dispense into tightly closed light protected glass bottles, preferably under nitrogen.

### Example 13

II. Postnatal Induction of Schizophrenia (Days 3-15) Followed by Oral Treatment of Adolescent Mice with BCP in SEDDS.

### Methods

Preparation of Diluted Oral SEDDS Vehicle with BCP for Administration by Gavage.

Sterile double-distilled water (DDW) was warmed for 10 mm in a pre-warmed thermobath (35-38° C.). The SEDDS vehicle of the oral composition of Example 1 was warmed up separately to 35-38° C. for 10 min. In order to prepare BCP (5 mg/ml) for a final dose of 10 mg/kg, BCP (5 mg) was added directly into the vehicle (1 ml) and vortexed for 1 min to obtain the oral composition. Then the warmed sterile DDW (4 ml) at 35-38° C. was added at a ratio of 1:5 oral composition: DDW dilution and the diluted composition was vortexed for 1 min. In order to prepare BCP for a final dose of 5 mg/kg, 500 μl of BCP at 5 mg/ml were diluted with 500 μl SEDDS vehicle (1:2 dilution). Then the warmed sterile DDW (4 ml) at 35-38° C. was added at a ratio of 1:5 oral composition. DDW dilution and the diluted composition was vortexed for 1 min.

BCP (5 mg/kg or 10 mg/kg in diluted self-emulsifying vehicle (Example 1)) was administered to adolescent mice (10 μl/g) by gavage twice a week (on Sunday and Wednesday) for 3 weeks (PND 43-62), a total of 6 injections. Control group and PCP-induced group received by gavage the oral formulation solution without the drug. After the final BCP injection, mice were tested is the open field test (PND 64-66), forced-swimming test (PHD 70-71) and social interaction test (PND 88-89).

### Forced-Swimming Test

Training was conducted for 6 min a day before the test. Each mouse was placed into a transparent glass cylinder filled with fresh water at 25° C. On the test day, the total duration/frequency of immobility and climbing was counted every 2 minutes for 6 minutes. An increase in frequency of climbing serves as an index of increased despair.

### Open Field Test

Each mouse was placed into the center of a clear open Plexiglas Chamber (40 cm*32 cm*30 cm) which its floor was divided to squares of 4 cm×5 cm. Testing was performed in the presence of a bright white light. Ambulation behavior was manually counted for 8 min and data were collected in 2 min intervals.

### Social Interaction Test in a Novel Environment

Each mouse was placed in a novel cage together with a nonaggressive intruder mouse, of the same species, same sex and a similar age. The interaction between the two mice was recorded for 10 minutes with EthoVision software (Noldus). Social interaction was defined by contact between the mice (tracking nose point). Reduced duration of contact behavior indicates on impairment in social interaction.

### Results

FIG. 1 shows that oral treatment with 5 or 10 mg/kg BCP in SEDDS oral formulation at adolescence reversed the effect of PCP on mice in the forced-swim test. These results show that BCP acts orally and that the SEDDS composition used is efficient for oral administration. These results show that BCP in oral SEDDS composition is effective in reversing depression-like behavior, supporting its use as a pharmaceutical drug for the treatment of mental diseases in which depression is one of the symptoms (like for example bi-polar/mania-depressive disorder, depression, anxiety, ADHD, Tourette syndrome, depression associated with neurodegenerative diseases, depression that leads to metabolic diseases).

FIG. 2 of this disclosure shows that oral treatment with 5 mg/kg BCP in SEDDS oral formulation at adolescence reversed the effect of PCP on activity of mice in the open field test. These results show that BCP acts orally and that the composition used is efficient for oral administration. Comparison of the results in FIG. 2 in this disclosure with the results in FIGS. 14C-E in the U.S. Patent Application No. US 2015-0051299, shows that BCP in oral SEDDS composition is effective at the same dose as it has been shown for i.p. route of administration, which is surprisingly good.

FIGS. 3A-B show that oral treatment with 5 mg/kg BCP in SEDDS oral composition at adolescence reversed the effect of PCP on mice in the social interaction test (3A) but did not affect their body weight (3B). These results show that BCP acts orally and that the composition used is efficient for oral administration. These results show that BCP in oral SEDDS composition is effective in reversing deficits in social interaction.

### Example 14

III. Postnatal Induction of Schizophrenia (Days 3-15) Followed by Oral Treatment of Adolescent Mice with BCP in Oil.

### Methods

### Preparation of Diluted Oral Formulation of BCP for Administration by Gavage

BCP was diluted in canola oil.

BCP (10 mg/kg diluted in canola oil) was administered to adolescent mice (PND 4362) by gavage twice a week (on Sunday and Wednesday) for 3 weeks, a total of 6 gavages. Control group and PCP-induced group received by gavage the oil vehicle. After the final gavage, mice were tested in the open field test (PND 59), forced-swimming test (PND 83) and social interaction test (PND 88-89).

### Forced-Swimming Test

Training was conducted for 6 min a day before the test. Each mouse was placed into a transparent glass cylinder filled with fresh water at 25° C. On the test day, the total dilation/frequency of immobility and climbing was counted every 2 minutes for 6 minutes. An increased immobility is an index of learning and habituation, therefore a positive behavioral adaptation with a stressful condition.

### Results

FIG. 4 shows that oral treatment with 10 mg/kg BCP in oil composition did not reverse the effect of 5 mg/kg PCP on the frequency of immobility of mice in the forced swim test. These results show that BCP in SEDDS composition is effective while BCP in oil was ineffective on reversing the frequency of climbing in the forced-swim test. These results show that BCP in SEDDS oral composition surprisingly work much better than other oral compositions.

### Example 15

Preparation of MH in SEDDS vehicle.
1. The SEDDS vehicle of the oral composition of Example 1 was warmed up separately to 42° C. for 13 min. In order to prepare about 5% (g/vol) MH solution, the vehicle (380 mg) was added to MH (22.8 mg) directly and vortexed for 60 sec to obtain the oral composition.
2. The SEDDS vehicle of the oral composition of Example 1 was warmed up separately to 52° C. for 15 min. In order to prepare about 5% (g/vol) MH solution, the vehicle (380.18 mg) was added to MH (21.2 mg) directly and vortexed for 100 sec to obtain the oral composition. Then the solution was warmed up to 50° C. for 10 min. Then the solution was warmed up to 57° C. for 13 min and vortexed for 180 sec.
3. The SEDDS vehicle of the oral composition of Example 1 was warmed up separately to 55° C. for 15 min. In order to prepare about 1% (g/vol) MH solution, the vehicle (396 mg) was added to MH (4 mg) directly and vortexed for 60 sec to obtain the oral composition. Then the solution was warmed up to 55° C. for 10 min and vortexed for 60 sec.

### Results

1. MH did not dissolve in SEDDS. Two phases were evident.
2. MH did not dissolve in SEDDS. Two phases were evident. Warming up to 50° C. did not dissolve the MH. Warming up to 57° C. dissolved the MH, and phases disappeared.
3. MH did not dissolve in SEDDS. However, MH dissolved in SEDDS after warming to 55° C., affording one homogeneous phase.

### Example 16

The solution of 4-O-methylhonokiol (MH) was prepared in oral formulation according to Table 1 below.

### Results

MH dissolved in V-01 or in V-02 or in V-03. No phases were evident.

TABLE 1

| | | | V-01 | | V-02 | | V-03 | |
|---|---|---|---|---|---|---|---|---|
| Component | Supplier | Cat. No. | mg | % | mg | % | mg | % |
| Medium chain triglycerides (MCT oil) | Lipoid | 940028/909 | 45,000 | 40.90% | 45,000 | 39.81% | [symbol] | [symbol] |
| DL-alpha Tocopherol acetate USP | Sigma | T3376 | 10,000 | 9.09% | 10,000 | 8.85% | 9,000 | [symbol] |
| DL-alpha tocopherol USP | Sigma | T3251 | 5,000 | 4.54% | 5,000 | 4.42% | 4,500 | 3.84% |
| Butylated hydroxytoluene | Sigma | 37450 | 25 | 0.028% | 25 | 0.022% | 25 | 0.021% |
| Polyoxyl 35 castor oil NF (Kolliphor ELP) | Sigma | 30906 | 35,000 | 31.81% | | | | |
| Tween-60 (Polysorbate 60 NF) | Sigma | 95754 | | | 36,000 | 31.83% | | |
| Tween-80 (Polysorbate 80 NF) | Sigma | 59924 | | | | | 35,000 | 29.86% |
| Span 80 (Sorbitan monooleate) NF | Sigma | 85548 | 10,000 | 9.09% | 12,000 | 10.62% | 6,200 | 5.29% |
| Tocophersolan (TPG5, Tocopherol PEG | Sigma | 57668 | | | | | 8,500 | 7.25% |
| ester succinate) | | | | | | | | |
| Labrafil M1944 CS | Gattefosse | 3063 | | | | | 14,000 | 11.94% |
| Lecithin (Phospholipon 80) | ALC | 228197 | 5,000 | 4.54% | 5,000 | 4.42% | | |
| Ethyl alcohol anhydrous | Commercial | N/A | 5,000 | 4.54% | 5,000 | 4.42% | | |
| | alcohols | | | | | | | |
| Total, mg | | | 110,025 | 100.00% | 118,025 | 100.00% | 117,225 | 100.00% |

[symbol]

indicates data missing or illegible when filed

### REFERENCES

Anavi-Goffer, S. G. Baillie, A. J. Irving, J. Gertsch, I. R. Greig, R. G. Pertwee, and R. A. Ross. (2011) Modulation of L-alpha-lysophosphatidylinositol/GPR55 MAP kinase signalling by cannabinoids. J Biol Chem. 287: 91-104.
Ballmaier M, Bortolato M, Rizzetti C, Zoli M, Gessa G, Heinz A, Spano P. (2007) Cannabinoid receptor antagonists counteract sensorimotor gating deficits in the phencyclidine model of psychosis. Neuropsychopharmacology. 32: 2098-2107.
Childers S R. Activation of G-proteins in brain by endogenous and exogenous cannabinoids. AAPS J. 2006;8;E112-E117.
De Marchi N, De Petrocellis L, Orlando P, Daniele F, Fezza F, Di Marzo V (2003) Endocannabinoid signalling in the blood of patients with schizophrenia. Lipids Health Dis. 2:5.
Di Marzo V, Bifulco M, De Petrocellis L (2004) The endocannabinoid system and its therapeutic exploitation, Nat Rev Drug Discov. 3:771-784.
Fride E, Gobshtis N, Dahan H, Weller A, Giuffrida A, Ben-Shabat S (2009) The endocannabinoid system during development: emphasis on perinatal events and delayed effects. Vitam Horm. 81:139-58.
Gamhi F, De Berardis D, Sepede G, Quartesan R, Calcagni E, Salerno R M, Conti C M, Ferro F M (2005) Cannabinoid receptors and their relationships with neuropsychiatric disorders. Int J Immunopathol Pharmacol. 18:9-25.
Gardner E L (2005) Endocannabinoid signaling system and brain reward: emphasis on dopamine. Pharmacol Biochem Behay. 81:263-284.
Gertsch J, et al. 2008. Beta-caryophyllene is a dietary cannabinoid. Proc Natl Acad Sci USA 105(26): 9099-9104.
Henstridge, C. M., N. A. Balenga, R. Schroder, J. K. Kargl, W. Platzer, L. Martini, S. Arthur, J. Penman, J. L. Whistler, E. Kostenis, M. Waldhoer, and A. J. Irving. (2010), GPR55 ligands promote receptor coupling to multiple signalling pathways. Br J Pharmacol. 160: 604-14.
Hashimoto K, Fujita Y, Shimizu E, Iyo M (2005) Phencyclidine-induced cognitive deficits in mice are improved by subsequent subchronic administration of clozapine, but not haloperidol. Eur J Pharmacol. 519: 114-117.
Josselyn S A and Vaccarino F J (1998) Preclinical behavioral approaches and study of antipsychotic drug action and schizophrenia, in in vivo neuromethods (Boulton A A, Baker G B and Bateson A N eds) pp 177-225, Humana Press, Totowa.
Leweke F M, Giuffrida. A, Wurster U, Emrich H M, Piomelli D (1999) Elevated endogenous cannabinoids in schizophrenia. Neuroreport. 10:1665-1669.
Long L E, Malone D T, Taylor D A. (2006) Cannabidiol reverses MK-801-induced disruption of prepulse inhibition in mice. Neuropsychopharmacology. 4:795-803.
Nagaraju J. M. pharmacy II Semester 2010. Department of Pharmaceutics, University College of Pharmaceutical Sciences, Kakatiya University, Warangal. http://documents.mx/documents/seminar-on-self-emulsifying-drug-delivery-system-by-jnagaraju-mpharmacy-ii.html
Newell K A, Deng C, Huang X F. (2006) Increased cannabinoid receptor density in the posterior cingulate cortex in schizophrenia. Exp Brain Res. 172:556-60.
Ortega-Alvaro, A., A. Aracil-Fernandez, M. S. Garcia-Gutierrez, F. Navarrete, and J. Manzanares. (2011) Deletion of CB2 cannabinoid receptor induces schizophrenia-related behaviors in mice. Neuropsychopharmacology 36:1489.
Takahashi M, Kakita A, Futamura T, Watanabe Y, Mizuno M, Sakimura K, Castren E, Nabeshima T, Someya T, Nawa H (2006) Sustained brain-derived neurotrophic factor up-regulation and sensorimotor gating abnormality induced by postnatal exposure to phencyclidine: comparison with adult treatment. J Neurochem. 99:770-780.
Turgeon S M, Kim D, Pritchard M, Salgado S, Thaler A (2011) The effects of phenylcyclidine (PCP) on anxiety-like behavior in the elevated plus maze and the light-dark exploration test are age dependent, sexually dimorphic and task dependent.
Varty G B, Walters N, Cohen-Williams M, Carey G J (2001) Comparison of apomorphine, amphetamine and dizoeilpine disruptions of prepulse inhibition in inbred and outbred mice strains, Eur J Pharmacol. 424:27-36.
Wang C Z & Johnson K M (2005) Differential effects of acute and subchronic administration on phencyclidine-induced neurodegeneration in the perinatal rat. J Neurosci Res, 81:284-292. Wiley J L, Cristello A F, Balster R L (1995) Effects of site-selective NMDA receptor antagonists in an elevated plus-maze model of anxiety in mice. Eur J Pharmaco. 294:101-107

1. A stable self-emulsifying composition for treatment of mental disorders in a patient in need thereof, comprising:
a therapeutically effective amount of at least one CB2 receptor modulator, wherein the at least one CB2 receptor modulator is selected from the group consisting of a CB2 receptor agonist or partial agonist, a CB2 receptor antagonist or inverse agonist, a CB2 receptor antagonist or inverse agonist that is a selective estrogen receptor modulator (SERM), a CB2 receptor allosteric modulator and combinations thereof,
a self-emulsifying vehicle, and
optionally a therapeutically effective amount of an active agent,
wherein the active agent comprises at least one antipsychotic agent,
at least one anti-inflammatory agent at least one GPR55 modulator, or combinations thereof,
wherein at least one CB2 receptor modulator and the optional at least one active agent are substantially solubilized.

2. (canceled)

3. (canceled)

4. The composition of claim 1, wherein the at least one CB2 receptor agonist or partial agonist is selected from the group consisting of BCP, HU-308, HU-433, HU-910, HU-914, CB 65, GP 1a, GP 2a, GW 405833, JWH 015, JWH 133, AM1241, L759,656, L-759,633, MDA 19, SER 601, BML-190, N-alkylamide, rutamarin, diindolylmethane (DIM), cannabinor (PRS-211,375), 2-arachidonoylglycerol, anandamide, CP55940, delta-9-THC, W1N55212-2, HU-210, cannabigerol (CBG), 11-hydroxy-Δ9-tetrahydrocannabinol (11-OH-THC), delta-8-THC, 11-OH-delta-8-THCV, ajulemic acid, delta-8-THC-11-oic acid, cannabinol (CBN), cannabilactones, AM1714, AM1710, analogs thereof, derivatives thereof, metabolites thereof and combinations thereof.

5. The composition of claim 1, wherein the at least one CB2 receptor antagonist or inverse agonist is selected from the group consisting of AM630, JTE-907, SR144528, COR170, 4-0-methylhonokiol, GS12021 (4-0-methylhonokiol analogue), cannabinol, 01238, 01184, cannabidiol (CBD), analogs thereof, derivatives thereof and combinations thereof.

6. The composition of claim 1, wherein the at least one CB2 receptor allosteric modulator is selected from the group consisting of dihydrogambogic acid, garcinolic acid, (-)-5′-dimethylheptyl-cannabidiol (DMH-CBD), analogs thereof, derivatives thereof and combinations thereof.

7. The composition of claim 1, wherein the at least one CB2 receptor modulator is selected from the group consisting of raloxifene, bazedoxifen, lasofoxifene, tamoxifen, afimoxifene, arzoxifene, ormeloxifene, toremifene, ospemifene and analogs thereof, derivatives thereof and combinations thereof.

8. The composition of claim 1, wherein the at least one antipsychotic agent is a butyrophenone type antipsychotic agent, an atypical antipsychotic agent or a combination thereof,
wherein the butyrophenone type antipsychotic agent is selected from the group consisting of haloperidol, droperidol, benperidol, trifluperidol, melperone, lenperone, azaperone, domperidone, butyrophenone, fluanisone, penfluridol, pipamperone, spiperone, nonaperone, bromperidol and timiperone, a diphenylbutylpiperidine type antipsychotic agent selected from the group consisting of luspirilene, penfluridol, pimozide, clopimozide, fluspirilene, penfluridol, a phenothiazine type antipsychotic acid agent selected from the group consisting of acepromazine, chlorpromazine, cyamemazine, dixyrazine, fluphenazine, levomepromazine, mesoridazine, perazine, pericyazine, perphenazine, pipotiazine, prochlorperazine, promazine, promethazine, prothipendyl, thioproperazine, thioridazine and trifluoperazine and triflupromazine, a thioxanthene type antipsychotic agent selected from the group consisting of chlorprothixene, clopenthixol, flupentixol, thiothixene and zuclopenthixol, and combinations thereof,
wherein the atypical antipsychotic agent is an atypical antipsychotic agent belonging to the D2 antagonist/inverse agonist or 5-HT2A antagonist/inverse agonist types and is selected from the group consisting of amisulpride, amoxapine, asenapine, cariprazine, clozapine, blonanserin, iloperidone, lurasidone, melperone, nemonapride, olanzapine, paliperidone, paliperidone palmitate, perospirone, quetiapine, remoxipride, risperidone, sertindole, sultopride, trimipramine, ziprasidone, ITI-007, pimavanserin (ACP-103; 5-HT2A antagonist), and combinations thereof,
wherein the atypical antipsychotic agent is an atypical antipsychotic agent belonging to the D2 partial agonist types and is selected from the group consisting aripiprazole and its metabolites OPC-14857, DM-1458, DM-1451, DM-1452, DM-1454 and DCPP, brexpiprazole and RP5063 (RP5000) and combinations thereof and/or a cannabinoid exhibiting antipsychotic activity selected from the group consisting of tetrahydrocannabivarin (THCV—CB1 antagonist, CB2 receptor partial agonist), cannabidiol (CBD—CB1/CB2/GPR55/ABn-CBD antagonist/inhibitor) and cannabigerol (CBG—CB1/CB2 partial agonist), and their analogs and derivatives and combinations thereof.

9. (canceled)

10. The composition of claim 1, wherein the composition is formulated as a stable self-emulsifying drug delivery system and wherein the composition comprises:
from 10% w/w to 50% w/w of an oil selected from the group consisting of medium chain triglycerides, propylene glycol dicaprilate/dicaprate, medium chain mono- and diglycerides, acetylated mono- and diglycerides, sesame oil and olive oil and combinations thereof,
from 20% w/w to 50% w/w of a surfactant HLB<9 selected from the group consisting of oleoyl polyoxyl-6 glycerides, linoleyl polyoxyl-6 glycerides (20-40%), Polysorbate 85 (Tween-85) polyoxyethylene (20-40% w/w), sorbitan trioleate (5-15% w/w), Span-80 (sorbitan monooleate) (5-25% w/w), polyglyceryl-3 dioleate (15-35% w/w) and glycerin monolinoleate (10-35% w/w), Polysorbate 80 (Tween-80) polyoxyethylene (20-40% w/w), Polysorbate 60 (Tween-60) polyoxyethylene (20-40% w/w), and combinations thereof,
from 5% w/w to 50% w/w of a surfactant HLB>13 selected from the group consisting of polyoxylated castor oil (5-40% w/w), PEG 40 hydrogenated castor oil, PEG-15 hydroxystearate (5-25% w/w), caprylocaproyl polyoxyl-8 glycerides (10-20%) w/w) and combinations thereof,
from 5% w/w to 25% w/w of a surfactant HLB>13 selected from the group consisting of PEG-20 sorbitan monostearate, PEG-20 sorbitan monooleate (5-25%), PEG 40 stearate (5-25% w/w) and combinations thereof, from 0.5% w/w to 15% w/w of a co-surfactant selected from the group consisting of any lecithin (2-15% w/w), soy lecithin (≥75% w/w phosphatidylcholine in oil, 1-10% w/w), soy lecithin PC content >50% (2-15% w/w), egg lecithin E-60 (1-5% w/w), egg lecithin E-80 (1-5% w/w), distearoylphosphatidylcholine (0.5-3%) w/w) and combinations thereof,
from 0.1%) w/w to 5% w/w of an antioxidant or free radical scavenger selected from the group consisting of d-alpha-tocopherol (1-10% w/w), dl-alphatocopherol (2-15% w/w), dl-alpha-tocopheryl acetate (2-15% w/w), mixed tocopherols (alpha, beta, gama—1-10% w/w), d-alpha-tocopheryl acetate (2-15% w/w), butylated hydroxyanisole (BHA, 0.01-0.5%) w/w), tocophersolan (TPGS, tocopherol PEG ester succinate) (2-10% w/w) and combinations thereof,
from about 1% w/w to about 10% w/w of ethyl alcohol,
from 1% w/w to 20% w/w of at least one CB2 receptor modulator in substantially pure form, and
optionally from 0.1% w/w to 5% w/w of at least one antipsychotic agent.

11. The composition of claim 10, wherein the composition is formulated as a stable self-emulsifying drug delivery system and wherein the composition comprises:
from 30% w/w to 50% w/w capric/caprylic triglycerides,
from 30% w/w to 50% w/w oleoyl polyoxyl-6 glycerides,
from 5% w/w to 35% w/w polyoxylated castor oil,
from 7% w/w to 15% w/w PEG-20 sorbitan monostearate,
from 2% w/w to 10% w/w soy lecithin (75% phosphatidylcholine in oil),
from 1% w/w to 15% w/w d-alpha tocopherol and/or tocopherol acetate,
from 1% w/w to 20% w/w of at least one CB2 receptor modulator, and
optionally from 0.1% w/w to 5% w/w of at least one antipsychotic agent.

12. (canceled)

13. The composition of claim 1, wherein the at least one CB2 receptor modulator is selected from the group consisting of beta-caryophyllene (BCP), HU-308, and 4-0-methylhonokiol (MH), and wherein the optional at least one antipsychotic agent is selected from the group consisting of risperidone, paliperidone, paliperidone palmitate, aripiprazole, quetiapine, CBD, THCV, CBG, brexpiprazole, derivatives thereof, analogs thereof and combinations thereof.

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. The composition of claim 1, wherein the composition is formulated for oral, parenteral, topical, intranasal, vaginal or rectal administration.

27. The oral composition of claim 1, wherein the composition is formulated as a spray, inhalation, capsule, suspension, solution, emulsion, depot injection, gel, cream, patch or syrup.

28. (canceled)

29. (canceled)

30. A method of treatment of a mental disorder in a patient in need thereof, by administration of the composition of claim 1.

31. A method of treatment of a mental disorder in a patient in need thereof, by administration of a composition comprising a therapeutically effective amount of at least one CB2 receptor modulator, wherein the at least one CB2 receptor modulator is beta caryophyllene (BCP), a self-emulsifying vehicle, optionally at least one active agent comprising alpha-humulene, copaene, eugenol, δ-cadinene, BCP oxide combinations thereof, and
optionally a therapeutically effective amount of at least one antipsychotic agent.

32. The method of claim 30, a wherein the at least one CB2 receptor selective agonist is selected from the group of HU-308, and 4-0-methylhonokiol (MH), and a substantially pure form of beta caryophyllene (BCP), and
optionally wherein the composition comprises at least one antipsychotic agent, wherein the at least one antipsychotic agent is selected from the group consisting of risperidone, paliperidone, paliperidone palmitate, aripiprazole, quetiapine, CBD, THCV CBG, brexpiprazole, derivatives thereof, analogs thereof and combinations thereof, and wherein the mental disorder is schizophrenia.

33. (canceled)

34. (canceled)

35. (canceled)

36. The method of treatment of claim 31, wherein the at least one antipsychotic agent is selected from the group consisting of risperidone, paliperidone, paliperidone palmitate, aripiprazole, quetiapine, CBD, derivatives thereof and analogs thereof, THCV, CBGV, brexpiprazole and combinations thereof.

37. (canceled)

38. (canceled)

39. (canceled)

40. (canceled)

41. (canceled)

42. (canceled)

43. The method of treatment of claim 30, wherein the at least one antipsychotic agent is selected from the group consisting of one or more of a butyrophenone type antipsychotic agent selected from the group consisting of haloperidol, droperidol, benperidol, trifluperidol, melperone, lenperone, azaperone, domperidone, butyrophenone, fluanisone, penfluridol, pipamperone, spiperone, nonaperone, bromperidol and timiperone, a diphenylbutylpiperidine type antipsychotic agent selected from the group consisting of luspirilene, penfluridol, pimozide, clopimozide, fluspirilene, penfluridol, a phenothiazine type antipsychotic acid agent selected from the group consisting of acepromazine, chlorpromazine, cyamemazine, dixyrazine, fluphenazine, levomepromazine, mesondazine, perazine, pericyazine, perphenazine, pipotiazine, prochlorperazine, promazine, promethazine, prothipendyl, thioproperazine, thioridazine and trifluoperazine and triflupromazine, a thioxanthene type antipsychotic agent selected from the group consisting of chlorprothixene, clopenthixol, flupentixol, thiothixene and zuclopenthixol and/or an atypical antipsychotic agent including, but not limited to one or more of an atypical antipsychotic agent usually belonging to the D2 antagonist/inverse agonist, 5-HT2A antagonist/inverse agonist types selected from the group consisting of amisulpride, amoxapine, asenapine, cariprazine, clozapine, blonanserin, iloperidone, lurasidone, melperone, nemonapride, olanzapine, paliperidone, paliperidone palmitate, perospirone, quetiapine, remoxipride, risperidone, sertindole, sultopride, trimipramine, ziprasidone, ITI-007, pimavanserin (ACP-103; 5-HT2A antagonist), and combinations thereof, and/or an atypical antipsychotic agent selected from the group consisting aripiprazole and its metabolites OPC-14857, DM-1458, DM-1451, DM-1452, DM-1454 and DCPP, brexpiprazole and RP5063 (RP5000) and combinations thereof and/or a cannabinoid exhibiting antipsychotic activity selected from the group consisting of tetrahydrocannabivarin (THCV—CB1 antagonist, CB2 receptor partial agonist), cannabidiol (CBD—CB1/CB2/GPR55/ABN-CBD antagonist/inhibitor) and cannabigerol (CBG—CB1/CB2 partial agonist), analogs thereof, derivatives thereof and combinations thereof.

44. A method of treatment of claim 30, wherein the disease or mental disorder is selected from the group consisting of schizophrenia, schizoaffective disorder, bipolar disorder I and II, unipolar disorder, multiple personality disorder, psychotic disorders, depression, psychotic depression, depressive disorders, major depressive disorder, stereotypic movement disorder, autism spectrum disorders, obsessive-compulsive disorder (OCD), bacterial-induced tic disorder, pediatric autoimmune neuropsychiatric disorders associated with (streptococcal) infections (PANDAS), chorea (Sydenham's chorea (SC), chorea minor, chorea gravidarum, drug-induced chorea), drug-induced repetitive behaviors, akathisia, dyskinesias, Wernicke-Korsakoff syndrome, Tourette's syndrome, tic disorders, epilepsy, anxiety disorders, autistic spectrum disorder, enuresis, addiction, withdrawal symptoms associated with addiction, Asperger syndrome, oppositional defiant disorder, behavioral disturbance, agitation, psychosis/agitation associated with Alzheimer's disease, psychosis associated with Parkinson's disease, psychosis associated with drug of abuse, psychosis associated with psychedelic drug abuse, LSD-induced psychosis, steroid-induced schizophrenia, steroid-induced psychosis, Capgras syndrome, Fregoli syndrome, Cotard, personality disorders, borderline personality disorder, avoidant personality disorder, attention-deficit/hyperactive disorder (ADHD, ADD, HD), mania, dementia, anorexia, anorexia nervosa, anxiety, generalized anxiety disorder, social anxiety disorder, body dismographic disorder, obsessive compulsive disorder, paranoid disorder, nightmares, agitation, post-traumatic stress disorder (PTSD), severe mood dysregulation, developmental coordination disorder, stereotypic movement disorder, bacterial-induced tic disorder, pediatric autoimmune neuropsychiatric disorders associated with streptococcal infections (PANDAS), chorea (Sydenham's chorea (SC), chorea minor, chorea gravidarum, drug-induced chorea), drug-induced repetitive behaviors, akathisia, dyskinesias, Wernicke-Korsakoff syndrome, neuroinflammatory diseases, neurodegenerative diseases, liver associated-diseases, hepatitis, alcohol-related liver disease, fibromyalgia, gastrointestinal diseases, inflammatory bowel disease, Crohn's disease, ulcerative colitis, cancer, depression or anxiety that leads to metabolic diseases, depression associated with any of the above clinical conditions, cognitive deficits associated with any of the above clinical conditions and combinations thereof, wherein the disorder is acute, transient or chronic disease.

45. (canceled)

46. (canceled)

47. (canceled)

48. (canceled)

49. The method of treatment of claim 30, wherein the composition comprises therapeutically effective amount of BCP, HU-308, 4-0-methylhonokiol (MH),or a selective estrogen receptor modulator that is selected from the group consisting of raloxifene, bazedoxifene, lasofoxifene, tamoxifen, afimoxifene, arzoxifene, ormeloxifene, toremifene, ospemifene, as sole active agent and a self-emulsifying vehicle, and wherein the composition is administered to a patient in need thereof from once a month to once every two months, to once every three months, to once every four months, to once every five months, to once every six months, to once per week, twice per week, 3 times per week, 4 times per week, 5 times per week, 6 times per week, once per day, twice per day, 3 times per day or 4 times a day.

50. The method of treatment of claim 49, wherein the average daily amount of BCP, HU-308, 4-0-methylhonokiol (MH), or a selective estrogen receptor modulator that is selected from the group consisting of raloxifene, bazedoxifene, lasofoxifene, tamoxifen, afimoxifene, arzoxifene, ormeloxifene, toremifene, ospemifene, administered in any daily mode of administration is in a range selected from the group consisting of 0.01-0.1 mg, 0.1-1 mg 1-10 mg, 10-25 mg, 25-100 mg, 100-1000 mg, or 100-3000 mg, according to the patient's age and the effectiveness of the composition.

51. (canceled)

52. (canceled)

53. (canceled)

54. (canceled)

55. (canceled)

56. The method of treatment of claim 31, wherein the at least one active agent is co-administered in a single dosage form together with said CB2 receptor modulator or wherein the at least one active agent is co-administered sequentially in a dosage form separate from said CB2 receptor selective agonist in either order.

57. (canceled)

58. (canceled)

59. The composition of claim 10, wherein the composition is formulated as a stable self-emulsifying drug delivery system and wherein the composition comprises:
from 0.01% w/w to 0.2% w/w butylated hydroxytoluene,
from 1% w/w to 40% w/w Tween-60 (Polysorbate 60 NF),
from 1% w/w to 40% w/w Tween-80 (Polysorbate 80 NF),
from 1% w/w to 15% w/w Span 80 (Sorbitan monooleate) NF,
from 1% w/w to 15% w/w Tocophersolan (TPGS, Tocopherol PEG ester succinate),
from 1% w/w to 30% w/w Labrafil M1944 CS,
from 1% w/w to 15% w/w Lecithin (Phospholipon 80),
from 1% w/w to 15% w/w Ethyl alcohol anhydrous, and,
optionally from 0.1% w/w to 5% w/w of at least one antipsychotic agent.

60. The composition of claim 1, wherein the composition is a delayed-release composition.
